(19) United States
(12) Patent Application Publication
(10) Pub. No.: US 2022/0054806 A1
(43) Pub. Date: Feb. 24, 2022

(54) METHODS, SYSTEMS, AND DEVICES FOR RELIEVING CONGESTION OF THE LYMPHATIC SYSTEM

(71) Applicants: Ghassan S. Kassab, La Jolla, CA (US); Sean Chambers, Bloomington, IN (US); Joshua Krieger, Topsfield, MA (US); Jillian Ivers, Brownsburg, IN (US)

(72) Inventors: Ghassan S. Kassab, La Jolla, CA (US); Sean Chambers, Bloomington, IN (US); Joshua Krieger, Topsfield, MA (US); Jillian Ivers, Brownsburg, IN (US)

(21) Appl. No.: 17/408,335

(22) Filed: Aug. 20, 2021

Related U.S. Application Data
(60) Provisional application No. 63/067,917, filed on Aug. 20, 2020.

(51) Int. Cl. A61M 27/00; A61M 60/295; A61M 60/841; A61M 60/50

(52) U.S. Cl. CPC A61M 27/002; A61M 60/50; A61M 60/841; A61M 60/295

(57) ABSTRACT

Systems, devices and methods for treating lymphatic congestion are disclosed. In one method, a balloon is placed at or near the veno-lymph junction. The balloon is inflated and deflation through cycles of slow inflation and rapid deflation. In another embodiment, an arteriovenous fistula is created near the veno-lymph junction. Alternate embodiments may also include axial pumps, stents, or balloons in combination with the fistula. These devices and methods create an acceleration of the blood flow past the lymphatic duct which reduces local pressure via the Venturi effect and according to the Bernoulli principle which facilitates lymph entering into the bloodstream.

### PRIORITY

The present patent application is related to, and claims the priority benefit of, U.S. Provisional Patent Application Ser. No. 63/067,917 filed on Aug. 20, 2020, the contents of which are hereby incorporated by reference in their entirety into this disclosure.

### BACKGROUND

The lymphatic system, known as the ‘third circulatory system,’ is a complex architecture of vessels comprised of a lymphatic capillary network. This lymphatic capillary network comprises an extensive network of distensible channels which parallel the vascular systems and then drain into the veins. The lymphatic system collects and transports excess tissue fluid and extravasated plasma protein, absorbed lipids, and other large molecules from the intestinal space back to the venous system (jugular and subclavian veins) via the thoracic duct (TD). In particular, and under normal physiologic conditions, the thoracic duct drains into the left subclavian vein, and the right lymphatic duct drains into the right subclavian vein. However, under pathologic conditions, there may be an outflow obstruction, constriction, or congestion. This congestion may be anatomic or restrictive in regard to increased outflow resistance due to high lymphatic drainage in the presence of, for example, congestive heart failure (CHF) or other venous insufficiencies

The lymphatic system plays a critical role in tissue homeostasis. In normal mammals, it is estimated that 40% of the total plasma protein pool and an equivalent fluid to the total plasma volume are returned to the blood (i.e., central circulation) through the TD each day at approximately 1 ml/min. Unlike the arterial and venous counterparts, the lymphatic system is much less characterized and hence provides enormous opportunities for discovery of novel diagnostics and therapeutics.

There are both diagnostic and therapeutic targets for TD interventions which were pioneered by Dr. Cope two decades ago (Cope, 1995; Cope et al, 1997). For the former, changes in flow pressure and composition of TD can aid differential diagnosis of various disorders such as metastatic cancer, intestinal tuberculosis, Whipple disease, hepatic cirrhosis, bacterial infections, parasites, fungi, etc. to name just a few. On the latter, there are three major classes of therapy via TD access: 1) Removal of excess fluid or decompression of lymphatic system, 2) Elimination of toxic substance dissolved in lymph, and 3) Depletion of cells circulating in the TD.

In view of the foregoing, the present disclosure includes disclosure to address the therapeutic targets, namely the decongestion of the lymphatic system, so to treat CHF and other disorders relating to the lymphatic system.

In acute or congestive heart failure conditions, the right heart pressures are elevated, as is the pressure at the subclavian vein, which is where lymph drains from the TD. Under these conditions, the lymph flow from the thoracic duct is reduced (due to the higher pressure in the subclavian), which causes undesirable congestion of lymph at the veno-lymph junction (i.e., of the lymphatic system). Specifically, the higher pressure in the subclavian vein causes increased lymph formation (primarily by the liver) and this lymph then flows into the TD, which carries the lymph toward the subclavian vein. However, the increased pressure in the subclavian vein (during heart failure) impedes the drainage/flow of lymph and results in localized lymph congestion, with the associated signs and symptoms, such as undesirable fluid retention leading to ascites in the abdomen, fluid accumulation in the pericardial sac surrounding the heart, renal failure, and pulmonary edema, for example.

Currently, treatment to relieve congestion of the lymphatic system is accomplished using pharmaceuticals, such as diuretics and/or vasodilators. For more advanced heart disease conditions, current treatments may include supplemental oxygen to assist in breathing, or hospitalization for invasive procedures to actively drain excess fluid from the body. It would certainly be desirable to improve treatment methods and relieve the undesirable symptoms of lymphatic congestion for patients.

Disclosed herein are devices, methods, and systems that locally reduce the pressure at the veno-lymph junction (i.e., the junction of the subclavian/central vein and the thoracic duct) to increase the TD lymph flow. A physical principle by which this local pressure reduction may be accomplished is known as the Venturi effect (which stems from Bernoulli's principle of conservation of energy). Disclosed herein are devices, methods, and systems which accomplish a Venturi effect (i.e., increasing flow velocity to decrease pressure) near the veno-lymph junction to enhance lymph drainage into the venous subclavian vein/circulation both acutely and chronically. It would further be desirable to treat patients using minimally invasive devices, methods, and systems which do not require an external pump, but instead alter blood flow conditions in situ, without the need to remove the patient's blood from their body. The minimally invasive devices, methods, and systems disclosed herein create the advantageous blood flow conditions to relieve lymph congestion in situ, thus improving the standard of care and patient recovery rates, while minimizing adverse risks to the patient during a procedure.

### BRIEF SUMMARY

The present disclosure describes systems, devices, and methods which utilize Bernoulli's principle to achieve a Venturi effect through the increase of local blood flow velocity, thereby reducing local pressure at a veno-lymph junction and facilitating the entry of lymph into the bloodstream. The systems, devices, and methods are herein are useful for increasing lymph flow from a thoracic duct such as in acute cases of congestive heart failure.

In one embodiment, a method for reducing pressure at a veno-lymph junction to increase lymph flow from a lymphatic duct and alleviate lymphatic congestion, comprises: inserting a balloon catheter into a patient and positioning the balloon near the patient's veno-lymph junction; slowly inflating the balloon via an inflation lumen coupled to an inflation and deflation source, wherein slow inflation locally accelerates blood flow velocity toward a patient's heart; rapidly deflating the balloon after the balloon is inflated to create a suction effect to draw lymph out of a lymphatic duct; and repeating a cycle of slowly inflating the balloon and rapidly deflating the balloon until lymph flow from the lymphatic duct has been increased to alleviate the lymphatic congestion at the veno-lymph junction.

In an exemplary embodiment, the vein is the subclavian vein and the lymphatic duct is the thoracic duct.

The step of inflation should avoid damaging the vein, such as through excess pressure. An embodiment of the method further includes the step of sizing the patient's vein or veno-lymph junction to size the balloon catheter to be no more than 1 mm greater than an interior diameter of the vein near the veno-lymph junction to avoid exerting excess pressure on the vein. Further, the step of slowly inflating the balloon is performed at a rate such that the local pressure does not rise.

The cycle of slowly inflating the balloon and rapidly deflating the balloon may be performed automatically by a programmed pump. The balloon can also be inflated and deflated in sync with the right heart.

In another embodiment for reducing pressure at a veno-lymph junction to relieve lymphatic congestion, the method comprises: forming an arterial-venous (AV) fistula upstream of the veno-lymph junction to locally accelerate blood flow through an arterial jet towards the heart; and wherein the formation of the AV fistula locally accelerates blood flow at the veno-lymph junction and induces a local pressure drop at the thoracic duct to facilitate lymph flow therefrom to relieve lymphatic congestion.

Additionally, a stent can be inserted into the subclavian vein near the veno-lymph junction. The inserted stent can have an axial flow pump disposed therein and operating the axial flow pump can create blood flow acceleration toward the heart to decrease pressure and increase lymph flow from the thoracic duct and relieve lymphatic congestion.

The inserted stent may be a covered stent having a short and narrow center section and wherein the covered stent is configured for placement within a patient's subclavian vein, upstream of the veno-lymph junction, to create a region of lowered venous pressure at the thoracic duct. The inserted stent may also have a side branch configured to cannulate the veno-lymph junction and an outlet positioned with the short, narrow, center section, wherein the covered stent is deployed over the veno-lymph junction itself.

In an exemplary embodiment, the AV fistula connects the carotid artery and one of either the jugular vein or the subclavian vein. The method of claim 7 wherein the AV fistula is connected immediately proximal veno-lymph junction. The diameter of the AV fistula is preferably 4 mm to 6 mm in diameter, but may vary.

In an exemplary embodiment of a system for reducing pressure at a veno-lymph junction to increase lymph flow from a lymphatic duct and alleviate lymphatic congestion, the embodiment comprises: a catheter having a balloon thereon sized for insertion near a patient's veno-lymph junction and configured for inflation via a user at a proximal end thereof; wherein the balloon is sized to be no more than 1 mm greater than an internal diameter a patient's subclavian vein near the veno-lymph junction to avoid exerting excess pressure on the subclavian vein; and wherein cycles of slow inflation and rapid deflation of the balloon locally accelerate blood flow velocity towards the heart during the inflation, and creates a suction effect during the deflation, to draw lymph out of the thoracic duct and alleviate lymphatic congestion.

The balloon may be a compliant or semi-compliant balloon having a single chamber and a consistent size or a multi-chamber balloon. The system may include a manual inflation and deflation source or may comprise a programmable and automatic inflation and deflation source which may be synced to the heart.

### BRIEF DESCRIPTION OF THE DRAWINGS

The disclosed embodiments and other features, advantages, and disclosures contained herein, and the matter of attaining them, will become apparent and the present disclosure will be better understood by reference to the following description of various exemplary embodiments of the present disclosure taken in conjunction with the accompanying drawings, wherein:

FIG. 1 illustrates images of polymer casts of TDs:

FIG. 2 illustrates images of healthy swine TD vessel histology;

FIG. 3 illustrates images of healthy swine TD vessel histology;

FIG. 4 illustrates a schematic of TD vessel pressure vs. diameter bench top test set up;

FIG. 5 illustrates a graph of transmural pressure vs. mean TD diameter ratio;

FIG. 6 illustrates a schematic of vessel flow bench top test set-up;

FIG. 7 illustrates a graph of mean flow rate vs. pressure gradient of TD lymphatic vessel segment compared to 2 mm and 3 mm silicone tubes;

FIG. 8A is an image of the tricuspid injury model of the right ventricle;

FIG. 8B is an image of the tricuspid injury model of the tricuspid leaflet at the septum;

FIG. 8C is an image of the tricuspid injury model of the tricuspid leaflet at the anterior;

FIG. 9 illustrates graphs of jugular vein pressure before and after tricuspid injury (i.e., baseline/normal; immediate post-injury; and 4 weeks post-injury) for Pig #2565;

FIG. 10A is an image of a TD as observed in an normal animal;

FIG. 10B is an image showing significant dilation of the TD as observed in an animal 12 weeks post-tricuspid injury;

FIG. 10C is an image showing edema around an aortic-TD sheath;

FIG. 11 illustrates a contrast image of transonic flow taken by a probe placed on TD of swine;

FIG. 12A is an image of a control TD;

FIG. 12B is an image of TD thickening 4 weeks post injury;

FIG. 12C is an image of TD thickening 6 weeks post injury;

FIG. 12D is an image of TD thickening 12 weeks post injury;

FIG. 13A is an immunofluorescent image of a control TD;

FIG. 13B is an immunofluorescent image of a TD 6 weeks post injury;

FIG. 13C is another immunofluorescent image of a TD 6 weeks post injury;

FIG. 13D is an immunofluorescent image of a TD 12 weeks post injury;

FIG. 14A illustrates a graph of diameter vs. pressure relationship;

FIG. 14B illustrates a graph of diameter ratio vs. pressure relationship;

FIG. 15A is an image of fluoroscopic localization of the TD;

FIG. 15B is an image of fluoroscopic localization of the jugular vein;

FIG. 15C is an image of IVUS localization of the lymphovenous junction;

FIG. 16A illustrates an image of balloon inflation in the jugular vein;

FIG. 16B illustrates an image of balloon deflation in the jugular vein;

FIG. 17 illustrates a graph of increase in TD flow during balloon inflation/deflation;

FIG. 18A is an image of localization of the lymphovenous junction and fistula position;

FIG. 18B is an image of localization of the jugular vein;

FIG. 18C is an image of localization of the arterio-venous fistula;

FIG. 19 illustrates a graph of increase in TD flow during A-V fistula;

FIG. 20 illustrates a deflated balloon near the veno-lymph junction; and

FIG. 21 illustrates an inflated balloon near the veno-lymph junction; and

FIG. 22 illustrates a stent having a pump disposed in the subclavian vein; and

FIG. 23 illustrates an arterial-venous fistula connected to the subclavian vein and a pump in the fistula; and

FIG. 24 illustrates a fistula connected to the subclavian vein;

FIG. 25 illustrates a stent placed near the veno-lymph junction; and

FIG. 26 illustrates a stent placed in the veno-lymph junction.

An overview of the features, functions and/or configurations of the components depicted in the various figures will now be presented. It should be appreciated that not all of the features of the components of the figures are necessarily described. Some of these non-discussed features, such as various couplers, etc., as well as discussed features are inherent from the figures themselves. Other non-discussed features may be inherent in component geometry and/or configuration.

### DETAILED DESCRIPTION

For the purposes of promoting an understanding of the principles of the present disclosure, reference will now be made to the embodiments illustrated in the drawings, and specific language will be used to describe the same. It will nevertheless be understood that no limitation of the scope of this disclosure is thereby intended.

The present disclosure includes disclosure relating to the first therapeutic class (i.e., decongestion of lymphatic system) with application to congestive heart failure (CHF) and other disorders.

The feasibility of thoracic duct (TD) lymph decompression/drainage has already been demonstrated in patients five decades ago (Dumont et al, 1963; Witte et al, 1969). Thoracic duct cannulation was made surgically in CHF patients (a total of 17 patients in two studies, mostly class IV stage) to allow drainage of the distended TD. The decompression therapy provided immediate resolution of a number of signs and symptoms, including significant reductions of the following: venous pressure, distention of veins, and peripheral edema. Ascites and hepatomegaly also diminished or resolved completely in those patients.

Despite the tremendous efficacy of this approach and relative safety, there are two major shortcomings, namely 1) required surgical access of TD, and 2) it only provides temporary relief as it does not address the root cause of lymphatic congestion. To reap a chronic therapeutic benefit, for example, the procedure must be repeated frequently. The first shortcoming has been addressed given the present non-surgical (percutaneous) access of the TD; however, a solution to the second shortcoming has previously not been addressed. The present disclosure addresses this second shortcoming, namely to provide a chronic therapeutic benefit previously unknown and unavailable in the medical arts.

To address this second shortcoming, it is important to determine what causes the bottleneck to drainage of lymphatic fluid into the venous system. This can only be answered by having an intimate understanding of the major determinants of lymphatic flow; namely: 1) resistance of lymphatic channels, and 2) the pressure gradient across the lymphatics. The former is dictated by the architecture (morphometry, branching pattern, etc.) and mechanical properties (passive compliance, active smooth muscle contraction, distribution of lymphatic valves, etc.) of the lymphatic system in health and in CHF. The latter requires an understanding of the hemodynamic conditions (pressure difference) between the lymphatic terminals and drainage veins.

Such an understanding has allowed for design solutions for decompression of the lymphatic system as included in the present disclosure. Specifically, creation of devices, systems, and methods for locally accelerating blood flow velocity (to decrease pressure as per the Venturi effect) and thus facilitate drainage of lymph from the TD, can address the second shortcoming noted above. As drainage of the lymphatic system to the venous system is critical (when dictated by a pressure gradient), such devices, systems, and methods as referenced in further detail herein, can provide the chronic relief needed to maintain a decongested lymphatic system.

In an exemplary embodiment, an elevated systemic venous pressure in CHF reduces the pressure gradient for lymphatic flow and a connection to a lower pressure venous system can increase/restore the pressure gradient. The requirements of any device, system, and method (diameter, lengths, opening/closing pressures, etc.), utilized at lymphatic and venous locations (e.g., TD-to-pulmonary vein given the lower pressure than the systemic veins where drainage normally occurs, Cole et al, 1967), etc., could only be determined once the above noted characterization of the lymphatic system are made.

Described herein are devices, methods, and systems that locally reduce the pressure at the veno-lymph junction or anastomosis (i.e., the junction of the subclavian/central vein and the TD) to increase TD lymph flow, utilizing the Venturi effect. The Venturi effect is accomplished through a local increase in the velocity of the blood flow, which then decreases the pressure, to conserve total energy according to Bernoulli's principle. Accordingly, the Bernoulli principle may be applied to accomplish a Venturi effect near the lymphovenous junction to enhance lymph drainage.

### EXPERIMENTAL DATA

Section I: Anatomy and Mechanical Properties

### A. Overview

During Phase I, the anatomical structure and mechanical properties of the TD were characterized in normal animals (acute studies). All animals used in testing were healthy domestic Yorkshire swine between 60 and 75 kg in weight. An index of the animals and completed experimental protocols is listed in Table 1. Experimental protocols were determined based on consensus need for statistical significance of bench, pressure, casting and flow data. Experimental studies on individual pigs were usually limited by time. For example, there was rarely enough time to conduct pressure, flow, and casting on the same animal. The in vivo TD pressure protocol alone can take 4-8 hrs. Hence, casting was usually omitted and conducted in only three out of eighteen swine.

TABLE 1
Phase 1 Animal Study Index

| | | | Completed | |
|---|---|---|---|---|
| Pig # | Pig ID | Date | Protocol | Notes |
| 1 | 1 | 3/24/2017 | Training | Flow planned, but could not find TD |
| 2 | 88988 | 5/31/2017 | Training / | Flow planned, but could not find TD. Tissue used for bench testing. |
| | | | Bench | |
| 3 | 195 | 6/15/2017 | Flow / | Open abdomen flow probe placement (superior to CC) and bench data |
| | | | Bench | |
| 4 | 161 | 6/21/2017 | Training | Failed transabdominal cannulation. Surgical exploration of TD anatomy. |
| 5 | 259 | 6/26/2017 | Pressure | Successful transabdominal cannulation. |
| 6 | 248 | 7/5/2017 | Bench | Failed transabdominal cannulation. Tissue used for bench testing |
| 7 | 223 | 7/10/2017 | Pressure | Successful transabdominal cannulation. |
| 8 | 409 | 7/17/2017 | Pressure | Successful transabdominal cannulation. |
| 9 | 427 | 7/26/2017 | Pressure | Successful transabdominal cannulation. |
| 10 | 499 | 8/2/2017 | Pressure | Successful transabdominal cannulation. |
| 11 | 1817 | 8/9/2017 | Training | Failed transabdominal cannulation. Animal used for surgical exploration |
| | | | | to attempt retrograde TD access approach. |
| 12 | 2146 | 8/30/2017 | Pressure / | Flow probe placed first, then transabdominal cannulation |
| | | | Flow | |
| 13 | 67366 | 9/15/2017 | Pressure | Successful transabdominal cannulation. |
| 14 | 2442 | 9/22/2017 | Pressure / | Flow probe placed first, then transabdominal cannulation. Casted. |
| | | | Flow | |
| 15 | 2520 | 10/5/2017 | Pressure / | Flow probe placed first, then transabdominal cannulation |
| | | | Flow | |
| 16 | 2462 | 11/16/2017 | Pressure/ | Flow probe placed first, then transabdominal cannulation. Only CC was |
| | | | Flow | measured for pressure. Abdomen was filled with fluid to simulate ascites. |
| 17 | 2487 | 12/4/2017 | Flow | Flow probe placed first, Abdomen was filled with fluid to simulate ascites. |
| | | | | Pressure and flow were observed during abdomen infusion. |
| 18 | 2680 | 12/21/2017 | Flow | Flow probe placed, but rate never changed, strange results and shape. |

### B. In Vivo Pressure and Flow Measurements

The in vivo TD pressure measurements were obtained using a trans-abdominal approach to cannulate the TD using a 2.8F Cantata microcatheter (Cook Medical) as described in the protocol document “PRO05JUL2017—Percutaneous access into the thoracic duct for delivery of diagnostics”. Once TD cannulation was confirmed using contrast fluoroscopy, a 0.014″ Primewire pressure sensing guide wire (Philips Volcano) was introduced into the vessel, advanced through the anastomosis, and into the vein just beyond the anastomosis. The mean pressure was measured at the vein outlet. The pressure sensor wire was withdrawn back through the TD and mean pressure values were recorded at the following 5 additional locations (for a total of 6): inside TD-vein anastomosis, at the level of the aortic arch, at the level of the heart apex, 10 cm caudal from the apex, and at the Cisterna Chyli (CC). Prior to use, each pressure sensing wire was calibrated within the range of 0-20 mmHg using a column of water.

In vivo TD pressure was recorded for nine of the eighteen acute animals studied in Phase 1. Those results are listed in Table 2. All of the Primewire pressure sensing wires used were determined to be accurate within 0.5 mmHg during calibration. The mean pressure gradient across the TD was 8.11 mmHg. This pressure gradient drives flow from the CC, antegrade through the TD and out through the primary TD lymphovenous anastomosis. This gradient was consistent in each of the animals (standard deviation=2 mmHg) despite a venous outlet pressure that varied from 4-15 mmHg. In some cases, measurements in the vein outlet were impossible as sharp angle of the lymphovenous anastomosis was impossible to cross—those instances are indicated with “None” in Table 2. Also, some TD pressure measurements were made without a radiopaque ruler available, making an accurate TD length measurement impossible; these cases are also indicated with “None”.

TABLE 2
TD Pressure in Healthy Pigs

| | | | | | | 10 cm | | | Length |
|---|---|---|---|---|---|---|---|---|---|
| | | Vein | TD-Vein | Aortic | Heart | Below | Cisterna | TD Δ Pressure | (Outlet |
| Pig # | Date | Outlet | anatomosis | Arch | Apex | Previous | Chyli | (CC − Outlet) | to CC) |
| 499 | Aug. 3, 2017 | 6 | 7 | 10 | 11 | 14 | 15 | 9 | 35 |
| 427 | Jul. 26, 2017 | 4 | 4 | 6 | 6 | 10 | 10 | 6 | 33 |
| 409 | Jul, 17, 2017 | 15 | 14 | 17 | 19 | 21 | 21 | 6 | 34 |
| 223 | Jul. 11, 2017 | 6 | 7 | 10 | 12 | 14 | 14 | 8 | 34 |
| 259 | Jun. 26, 2017 | 12 | 13 | 16 | 17 | 17 | 18 | 6 | 39 |
| 2146 | Aug. 30, 2017 | 5 | 5 | 9 | 11 | 16 | 16 | 11 | None |
| 67366 | Sep. 15, 2017 | None | 5 | 8 | 9 | 12 | 14 | 9 | 38 |
| 2442 | Sep. 22, 2017 | None | 4 | 9 | 11 | 13 | 15 | 11 | None |
| 2520 | Oct. 5, 2017 | 8 | 9 | 12 | 13 | 13 | 15 | 7 | 36 |
| Sample Set | Mean | 8.00 | 7.56 | 10.78 | 12.11 | 14.44 | 15.33 | 8.11 | 35.57 |
| Statistics: | Standard Deviation | 4.0 | 3.7 | 3.6 | 3.9 | 3.2 | 3.0 | 2.0 | 2.2 |
| | Standard Error | 1.53 | 1.25 | 1.21 | 1.31 | 1.07 | 1.00 | 0.68 | 0.74 |

TD flow was measured in vivo using a 2PS flow probe (indicated for acute use in vessels 1.5-2.0 mm OD; Transonic) placed around the TD at the level of the heart apex. The sizes of the flow probes were chosen based on literature data and protocols, also shown in Table 5. The TD was accessed via a right lateral thoracotomy. A 1-2 cm length of the TD vessel was carefully dissected away from the aorta and surrounding fascia. The probe was placed around the isolated TD vessel and the probe was surrounded as well as filled with ultrasound gel to obtain a good signal. The chest was closed using towel clamps and sealed using ultrasound gel. The animal was returned to supine position. Flow was measured prior to TD cannulation for pressure measurements. Flow was recorded continuously after placement, but the reported mean flow rate for each animal is based on a time average over the longest undisturbed time (i.e., when pig was supine and not being otherwise manipulated for any reason). This undisturbed period (stated as mean flow period in Table 3) ranged from 8-30 minutes, depending on the animal.

In vivo flow rates were measured in four acute animals. Those results are listed in Table 3. Measured flow rates were highly dependent on flow probe positioning. It was difficult to ensure that the probe remained in optimal position (perpendicular to TD vessel with lumen centered in probe ring) after closing the chest. The motion of the lungs and diaphragm can move the probe from its optimal perpendicular positioning and affect flow measurements. These challenges may have contributed to the variability observed in the in vivo flow data. The recorded flow rate data ranged from 0.23 to 1.52 ml/min for any 2-minute interval average in the undisturbed period. The mean flow rates recorded over the entire undisturbed time for each animal ranged from 0.42 to 1.44 ml/min.

TABLE 3
TD Flow Rates in Healthy Pigs

| | | Mean | Mean | Max 2 | Min 2 |
|---|---|---|---|---|---|
| | | Flow | Flow | min time | min time |
| | | Rate | Period | average | average |
| Pig # | Date | (ml/min) | (mins) | flow rate | flow rate |
| 2146 | 8/30/2017 | 1.44 | 8 | 1.52 | 1.3 |
| 2442 | 9/22/2017 | 0.46 | | | |
| 2462 | 11/16/2017 | 0.42 | 10 | 1.05 | 0.23 |
| 2487 | 12/4/2017 | 0.44 | 30 | 1.07 | 0.31 |
| Sample Set | Mean | 0.69 | | | |
| Statistics: | Standard Deviation | 0.50 | | | |
| | Standard Error | 0.25 | | | |

### C. Anatomical Characterization

TD anatomical dimensions were measured by creating polymer casts of the TD lumen. After the animal was euthanized, an afferent vessel of the CC was accessed via laparotomy and cannulated using the Seldinger technique. An introducer was advanced 3-5 cm into the vessel and a ligature was tied around the cannulated vessel to secure the cannula. 30-50 ml of the liquid two-part, catalyst-curing polymer (either MICROFIL® silicone or Batson's acrylic polymer casting materials) was injected into the TD through the introducer cannula using a syringe. The material is injected as a liquid with syringes and allowed to set at atmospheric pressure to avoid any changes to diameter of the vessels. After 45 minutes, the casting liquid polymerizes to a solid form that is later dissected out of the tissue. The resulting structure is a cast of the TD vessel lumen that can be studied and measured. These polymer casts were used to characterize the anatomical structure of the TD, to measure vessel diameter at several locations along the length, and to determine the average length of vessel between valves.

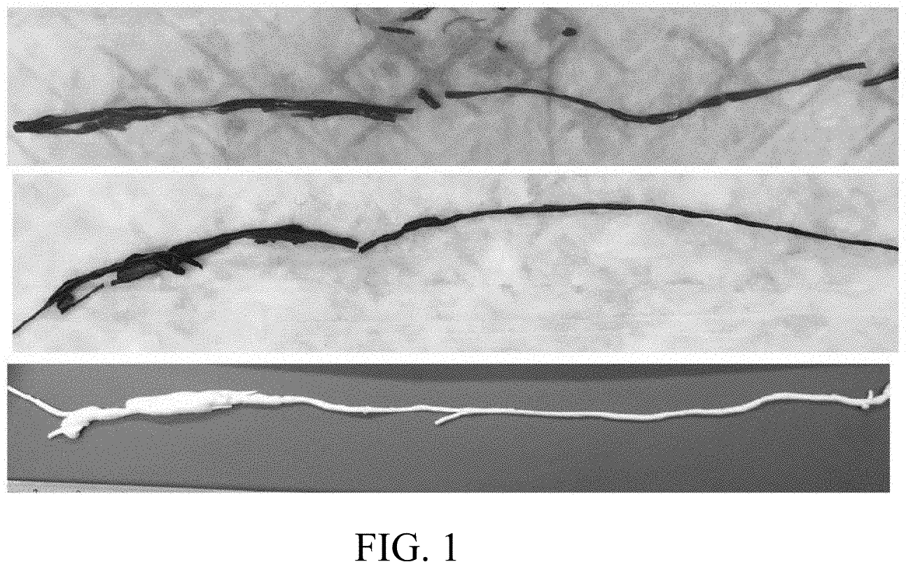

Polymer casts were created for three healthy animals. Pictures are shown in FIG. 1 and the measurements of the casts are listed in Table 3. A single measurement was recorded for the vessel diameter when cross section was circular. In other cases, multiple measurements were done and the mean was recorded. In most of the cases, the cross section was close to being circular. The cast lengths are not necessarily equal to the complete TD length, as the casting material did not always flow through the entire TD vessel. The CC structures of the cast were elongated vessels which wrapped around the descending aorta (the imprint of the aortic vessel is evident in each of the CC casts). The CCs appeared to be irregular structures that were consistently 2-3× wider than the main TD trunk. From the CC, the efferent TD vessel tapers from a wider profile at the CC to a smaller diameter in the main TD trunk. Bifurcations of the TD vessel were observed in fluoroscopy images but were not captured in any of the casts of healthy animals. The bifurcations were located at the level of the heart and created parallel paths that recombined again forming a single trunk prior to the main TD lymphovenous anastomosis. The final two measurements are approximately collocated with the heart apex and 10 cm below the heart apex locations used in the TD pressure measurements. The casts did not reach the level of anastomosis in most cases and hence, has not been reported.

Polymer casts of TDs are shown in FIG. 1. Each cast is oriented with the CC on the left, and lymphovenous anastomosis on the right. The topmost vast is an acrylic cast of Pig 88173. Segments of cast material above the main cast structure are from small branches and minor lympovenous anastomoses. The center cast is an acrylic cast of Pig 47489. This cast was incomplete as the material did not flow through the entire TD length. The bottom cast is a silicone cast of larger (100 kg) pig with ischemic HF from another protocol. Dimensions of this cast are not included in the data set, but the image is included here because it was the most complete cast obtained. The cast of Pig 2442 was damaged before being photographed.

TABLE 4
TD Polymer Cast Anatomical Dimensions of Healthy Pigs

| | | | | Diameter (mm) | | | | |
|---|---|---|---|---|---|---|---|---|
| | | Valve measurements | | | | | 10 cm | 10 cm |
| | | | Mean | | | | cranial | cranial |
| | | # Valves | length/valve | | | | from | from |
| Pig # | Cast Length (mm) | Total | (mm) | CC Max | CC Min | TD @ CC | previous | previous |
| 88173 | 18 | 9 | 2.0 | 15 | 2.7 | 3.6 | 2.2 | 2.1 |
| 2442 | 34 | 13 | 2.6 | 11.4 | 3 | 4.3 | 2.6 | 2 |
| 47489 | 28 | 9 | 3.1 | 12.9 | 2 | 2 | 2.2 | 2.3 |
| Sample Set | Mean | 10.3 | 2.6 | 13.1 | 2.6 | 3.3 | 2.3 | 2.1 |
| Statistics: | Standard Deviation | 2.3 | 0.6 | 2.5 | 0.2 | 0.5 | 0.3 | 0.1 |
| | Standard Error | 1.3 | 0.3 | 1.5 | 0.1 | 0.3 | 0.2 | 0.0 |

### D. Histology

TD vessel samples were dissected from the portion of TD adjacent to the descending aortas at level of the heart of healthy pigs and fixed in 4% paraformaldehyde for overnight and subsequently sectioned with a cryotome for histology. We stained samples with H&E to observe the vessel microstructure and with immunofluorescence (IF) probes to evaluate the cellular structures. The sections were processed for immunofluorescence procedures; i.e., blocking, permeabilization, primary antibodies incubation, and fluorescence secondary antibodies incubation. The primary antibody anti-smooth muscle alpha-actin (Abcam) was used to bond the alpha-actin of lymphatic smooth muscle cells in TD and fluorescence secondary antibody (Alexa Fluor 546) rendered visible fluorescence (red). DAPI was used to visualize cell nuclei (blue). The fluorescence microscope (Eclipse 200, Nikon) was used to obtain the images.

Images of the histology studies show that TD are thin walled vessels that are approximately 100 μm thick (n=5). The TD vessel walls typically have 1-2 layers of smooth muscle cells (SMC) (n=10) only. All the samples were from TD at level of the heart. No regional variation was observed along length of TD. As a limitation, we could not do any histology on the samples of the CC since they were difficult to dissect. Only length of TD along the descending aorta could be explanted for histology.

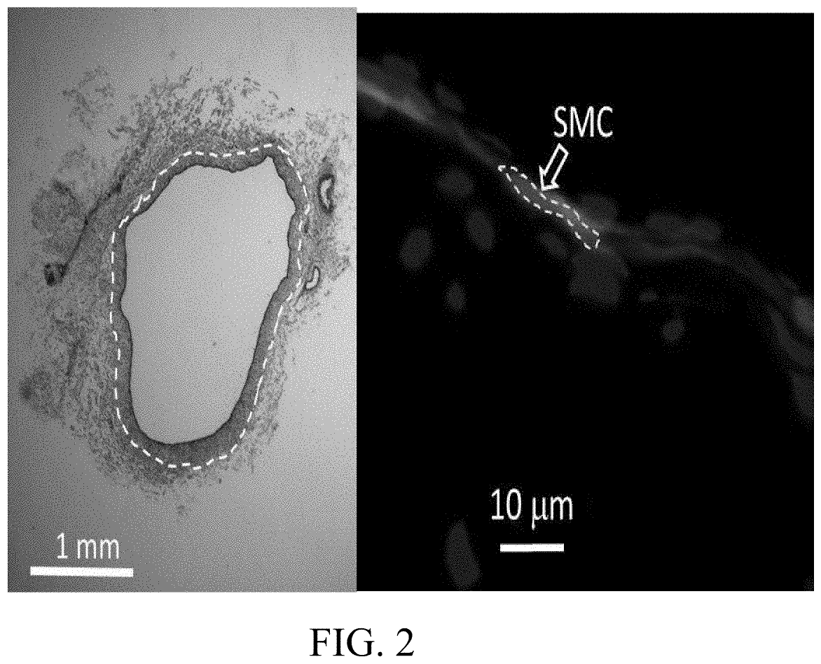

FIG. 2 displays images of healthy swine TD vessel histology. The H&E stained sample (Left) at 1× objective shows a vessel wall of approximately 100 μm. The IF stained samples show a single layer of SMC cells, which is the dominant structure for these sections. Red: smooth muscle alpha-actin. Blue: nuclei.

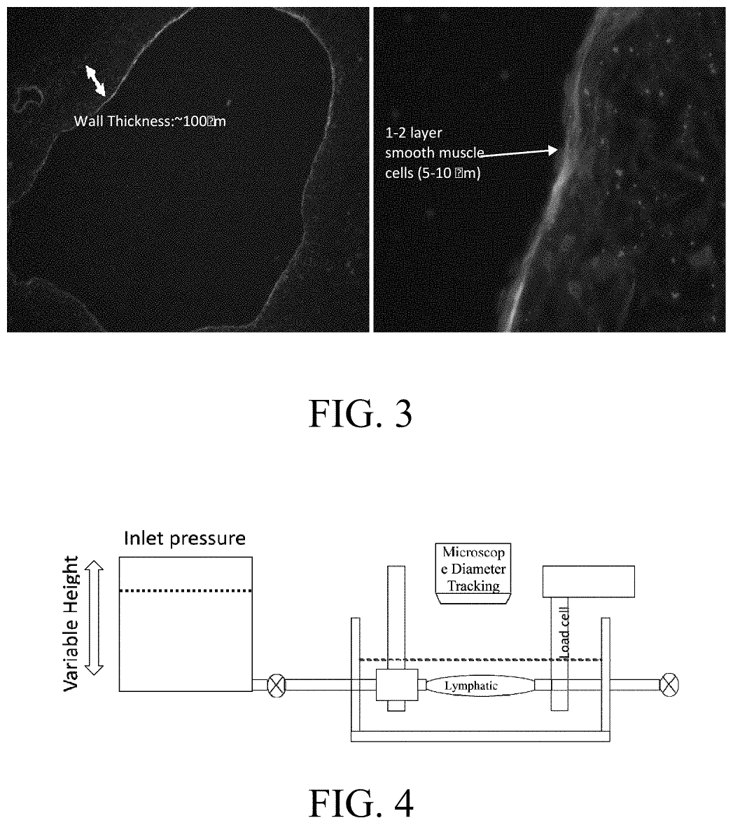

FIG. 3 comprises additional images of healthy swine TD vessel histology. The sample is stained with antibodies for smooth muscle alpha actin (green), nuclei (blue), collagen (red). The sample is shown at 4× objective (Left) and 60× objective (right).

### E. Mechanical Testing

Explanted sections of dissected TD vessels were used in bench top testing to characterize the passive mechanical pressure vs. diameter relationship of the vessels. The explanted TD samples (˜3 cm long) were taken from the section of TD adjacent to the descending aortas (3 TD samples of each pig were tested) of healthy pigs. The test setup consists of the cylindrical TD vessel cannulated on both ends to luers which were connected to containers using Tygon tubing and submerged an organ bath. The organ bath and vessel were filled with calcium-free PSS with 2.5 mmole/L EGTA 1 to completely relax the smooth muscle cells and stop any vasoactivity. The TD segment could be pressurized by raising a container which connected to TD segment through Tygon tubing and meanwhile blocking the other container. The pressure in TD segment was determined by the height of the container above the mounting points of the TD. The height of the container increased 1 cm by 1 cm up to 15 cm and jumped to 20 and 30 cm. The image of the TD segment was displayed on screen with a CCD camera mounted on a stereo microscope and the diameter change is measured with dimensional analysis software (DIAMTRAK 3+, Australia). The setup for the experiment is given in FIG. 4.

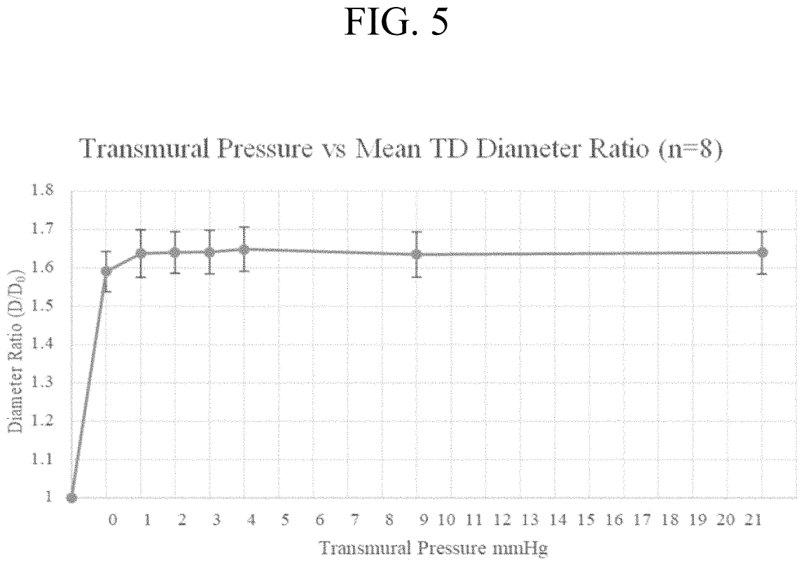

The pressure-diameter relationship was measured in samples from eight healthy pigs. The initial diameter (D0) value is taken as the diameter measured with no difference between the internal and exterior pressure of the cannulated vessel. When the transmural pressure (Internal pressure—external pressure) is set equal to 1, the mean diameter ratio was 1.59. The mean diameter ratio did not exceed 1.65 up to a transmural pressure of 20 mmHg (data was not recorded for transmural pressures exceeding 20 mmHg but from literature and plot in FIG. 5, it can be safely assumed that the mean diameter ratio will remain same).

### F. Pressure-Flow Relationship in the Thoracic Duct

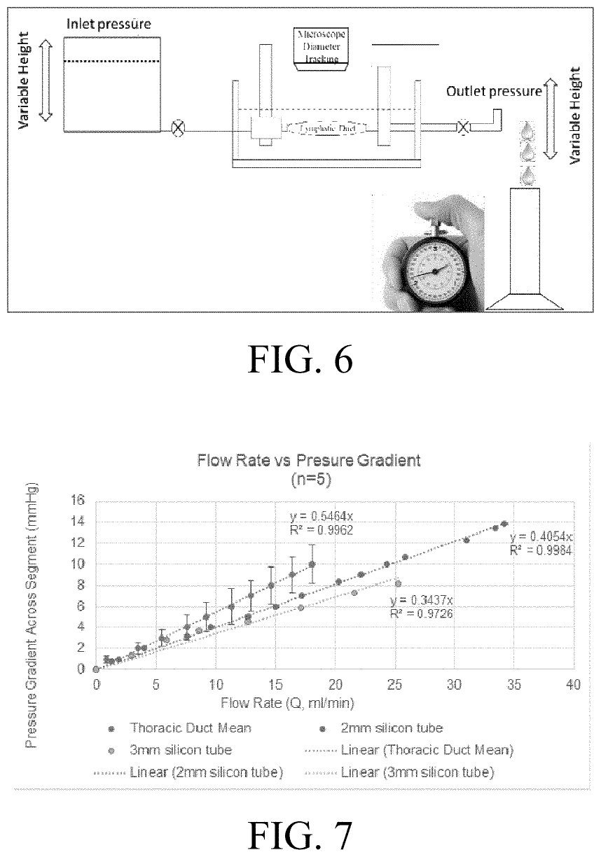

The bench top setup used to measure the pressure-flow relationship in TD samples was similar to the setup used to measure the pressure-diameter relationship. Explanted samples of TD vessels (3 cm long) were cannulated on both ends to luers which were connected to containers using Tygon tubing and submerged in an organ bath of with calcium-free PSS with 2.5 mmole/L EGTA 1 to completely relax the smooth muscle cells and stop any vasoactivity. The height of the container at outlet was adjusted to the same level of the organ bath. The height of the container at inlet was increased from the same level of organ bath to 15 cm above organ bath by 1 cm step, i.e., a pressure gradient across the TD segment was established by raising the fluid container at inlet. The inlet reservoir container is a large wide container such that during flow, there is negligible change in the water height and therefore, inlet pressure. The TD segment was oriented such that the flow was antegrade with the natural direction within the TD. Flow was determined by collecting the outlet flow in a graduated cylinder and measuring the volume collected over a time range of 30 sec to 3 min. The time range varied due to differences in inlet pressures which affected the flow rate. This test setup is depicted in FIG. 6. To estimate the resistance of the tubing system, a silicone tube (diameter 2 cm or 3 cm and 3 cm long) replaced the TD segment and the pressure-flow relation of silicone tubes represented the system resistance.

Results are shown in FIG. 7. The pressure-flow relationship was measured in samples from five healthy pigs. The mean diameter of the TD samples used was D_{O}=2.7 mm. The resistance of the vessel (Pressure Gradient/Flow Rate) was higher than in silicone tubes of 2 and 3 mm diameter. By subtracting the resistance of the system observed using the control silicone tubes, we can estimate the resistance associated with the vessel itself—attributed to valves and any other inner lumen surface features. The interpolated resistance of a 2.7 mm diameter silicone tube in the experimental system is 0.38. The mean resistance of the TD vessels in the experimental system is 0.56. Subtracting the resistance of a 2.7 mm silicone tube leaves the estimated isolated resistance of the lymphatic vessels: 0.56-0.38=0.17 (mmHg/ml/min). Reversing the pressure across the TD vessel samples resulted in a flow rate of 0 ml/min up to pressure gradients of 20 mmHg (highest recorded pressure) as the 1-way valves effectively blocked any flow.

### G. Discussion

The variety of experiments conducted within Phase 1 establishes a broad data set which describes the structure and function of the TD in healthy pigs. These data will provide input parameters and define the boundary conditions of the computational model to predict TD flow. They will also provide a baseline for comparison against data for animals with congestive heart failure (CHF).

To our knowledge, the in vivo TD pressure profile measurements are novel. Other researchers have measured pressure in the TD by cannulating the TD through the lymphovenous anastomosis [6-8], but there are no published data of pressure profile along the length of the TD down to the CC. These novel data were enabled by combining modern clinical techniques for TD access (transabdominal percutaneous approach) and guidewires with the use of solid-state pressure sensors (Philips Volcano Primewire) that are typically used for Fractional Flow Reserve (FFR) evaluation in patients with coronary ischemia. The consistent pressure gradient observed in the animals (8.11 mmHg+/−0.68) despite the wide range of outlet venous pressures (4-15 mmHg) suggests that compensatory feedback mechanisms in the lymphatic system can modulate pressures to achieve a consistent gradient across the TD to maintain normal flow rates against a healthy range of outlet pressures. In some cases, TD cannulation procedure required multiple punctures and sometimes a puncture of the CC. It is unclear the impact of this, but it seems likely that it would reduce the measured pressures, at least within the CC.

The anatomical course, features, and dimensions of the TD observed using the polymer casting methods are consistent with observations from literature. Although there is a wide variety of TD courses observed in large studies of patients, the most common course observed in humans (60%) [9] is a single main TD vessel running along the aorta from the CC to the lymphovenous anastomosis in the jugular vein. This was the same typical course observed in our animal studies. The shape of the CC, undulating diameter of the TD, and location of the main lymphovenous anastomosis were also similar to those observed in human patients [9]. Finally, the diameter of the cervical TD in humans has been observed to be 2.5 mm in healthy humans with an interquartile range of 1.8 mm to 3 mm [10]. These anatomical similarities suggest that domestic swine are a good model of lymphatic structure at least in terms of dimensions. Although the anatomical casting data agree with data from literature, more samples are required to obtain statistical confidence in a comparison with anatomical data from pigs with CHF. Casting was attempted on at least 7 healthy animals, but establishing and maintaining a reliable cannulation of the TD to inject the casting material can difficult. If the vessel is punctured or the cannula slips out, the liquid casting material does not flow into the TD prior to polymerization.

The thoracic duct flow rates recorded in the Phase 1 data set are lower than expected when compared with data from other researchers. Table 4 lists the published TD flow rates measured using Transonic flow probes. It is not clear why our flow rates have been lower than those reported in literature for other animal studies and including pigs. Our methods for placing the flow probes, recording, and interpreting flow data are consistent with the reported methods in these papers. More research is required to either establish confidence in our results or to determine the reason for our low flow rates.

The passive pressure-diameter relationship of TD vessels shows that they reach full capacity with only small increases in pressure, and once they reach their nominally full diameter, the vessels do not stretch to a larger size. The TD vessel sections reached a maximum diameter in response to only 1 mmHg transmural pressures and did not stretch significantly at higher pressures up to 20 mmHg. This relationship creates a system without the volumetric capacity that is observed in veins or even arteries which have more compliant vessel walls.

TABLE 5
TD Flow Rate Data from Literature Using Flow Probes

| | Baseline | | | | | | | | |
|---|---|---|---|---|---|---|---|---|---|
| | TD Flow | | | | | | | | |
| | Rate | Animal | Weight | Sample | Probe | Probe TD | Animal | | Time |
| Reference | (ml/min) | Model | (kg) | Size | Type | Location | Position | Status | Average |
| Onizuka et | 2.7 ± 1.9 | Sheep | 40 ± 4 | 7 | 2SB | 1 cm | | Anesthetized | |
| al. 1997 | | | | | or | cranial to | | | |
| | | | | | 3SB | median | | | |
| | 5.4 ± 3.1 | | 63 ± 11 | 4 | 2SB | mediastinal | | Awake | |
| | | | | | or | lymph | | | |
| | | | | | 3SB | node | | | |
| Inagaki et al. | 4.1 ± 1.3 | Sheep | 48 ± 9 | 6 | 2SB | 3-5 cm | | Anesthetized | |
| 2000 | | | | | | cranial to | | | |
| | | | | | | median | | | |
| | | | | | | mediastina | | | |
| | | | | | | 1 lymph | | | |
| | | | | | | node | | | |
| Tomoyasu | 2.75 ± 0.37 | Sheep | 49 ± 2 | 11 | H2SB | 7th | | Anesthetized | 5 min |
| et al. 2002 | | | | | | intercostal | | | |
| Knott et al. | 1.57 ± 0.2 | Dog | | 5 | 2SB | 4th | Standing | Awake | 3 min |
| 2005 | | | | | or | intercostal | | | |
| | | | | | 2.5SB | | | | |
| Lattuada & | 2.5 ± 0.4 | Pig | 28.8 ± 3 | 18 | 2.5SB | Caudal to | Supine | Anesthetized | |
| Hedenstiern | | | | | | diaphragm | | | |
| a 2006 | | | | | | | | | |
| Downey et | 1.7 ± 0.5 | Dog | | 8 | 2SB | 4th | Standing | Awake | 3 min |
| al. 2008 | | | | | or | intercostal | | | |
| | | | | | 2.5SB | | | | |

*Blank values are unreported

The resistance to flow in the TD is small in the antegrade direction and virtually infinite in the retrograde direction (up to at least 20 mmHg). This is consistent with observations from other researchers[17]. This relationship occurs because the valves in the TD are extremely efficient and can open and close in response to small changes in pressure. While the antegrade flow resistance is small, it does accumulate over the length of the TD. Using a networked model of lymphatic flow, Moore et al. calculated that the optimal number of valves along a lymphatic vessel length balances antegrade resistance with valve pumping power which results in maximum achievable flow rates. These data will be a critical input to the computational model for predicting flow in the TD in response to varying boundary conditions and pressures.

Section II: Chronic, Large Animal Model Development and Characterization of Remodeling

### A. Overview

The objective of Phase II was to establish an animal model of Congestive heart failure (CHF). CHF is one cause of ascites which is the accumulation of fluid within the peritoneal cavity. Congestion, or fluid overload, is a classic clinical feature of patients presenting with CHF patients, which is the commonest cause for hospitalization. The discomfort of swollen legs and ascites precipitates hospitalization. Congestion is associated with the sensation of breathlessness and reduces hepatic function. Congestion also causes renal dysfunction by reducing the trans-renal pressure gradient. Symptoms of CHF are relieved by removing excess fluid from the body, improving blood flow and heart muscle function; and increasing delivery of oxygen to the body tissues. The management of congestion in CHF is designed to improve cardiac function and to inhibit the hormonal and neurohumoral pathways that promote congestion.

Tricuspid regurgitation is one of etiology and pathogenesis of CHF. A swine model of tricuspid regurgitation (TCR) is developed in this project. TCR results in venous hypertension and increase in venous systemic pressure which drains more interstitial fluid into lymphatic system, i.e., fluid overload (congestion) in lymphatic system. An index of the animals and completed experimental protocols are listed in Table 6. Many of the methods detailed in Section I (for Phase I) were utilized. Novel methods are explained.

TABLE 6
Phase 2 Animal Study Index

| Pig # | Pig ID | Date | Completed Protocol | Notes |
|---|---|---|---|---|
| 1 | 2324 | 9/26/17 | 4wks post injury, pressure gradient, casting | |
| 2 | 2526 | 10/9/17 | 4wks post injury, pressure gradient, casting | |
| 3 | 2564 | 10/13/17 | 4wks post injury, pressure gradient | Casting failed |
| 4 | 2498 | 10/30/17 | 6wks post injury, pressure gradient | Partial casting |
| 5 | 2496 | 11/3/17 | 6wks post injury, pressure gradient | Partial casting |
| 6 | 2693 | 11/14/17 | 6wks post injury, pressure gradient, bench P-D, histology | |
| 7 | 3661. | 2/20/18 | 12wks post injury, pressure gradient, bench P-D, histology | |
| 8 | 3793 | 2/21/18 | 12wks post haiury, pressure gradient, bench P-D, histology | |
| 9 | 3823 | 5/22/18 | 12wks post injury, how probe, bench P-D, histology | Pressure gradient |
| 10 | 5018 | 7/11/18 | 12wks post injury, flow probe | failed |
| 11 | 9022 | 7/24/18 | Terminal study, histology | Flow probe failed |
| 12 | 5884 | 8/27/18 | 12wks post injury, flow probe | Flow probe failed |
| 13 | 6025 | 11/09/18 | 4wks post injury, pressure gradient | |
| 14 | 6026 | 11/14/18 | 4wks post injury, pressure gradient | |
| 15 | 6078 | 11/15/18 | 4wks post injury, pressure gradient | |
| 16 | 6323 | 12/05/18 | 4wks post injury, pressure gradient, chronic flow probe | |
| 17 | 6330 | 12/06/18 | 4wks post injury, pressure gradient, chronic flow probe | |
| 18 | 5331 | 12/10/13 | 4wks post injury, pressure gradient, chronic flow probe | |

### B. Development of CHF Swine Model

Tricuspid regurgitation was created by advancing a catheter and guidewire from the jugular sheath to the right atrioventricular junction under fluoroscopic guidance. A sheath (9F) was inserted through the jugular vein up to the right atrium (RA). The right ventricle (RV), RA, and jugular vein (JV) pressures were measured by inserting 5F catheter through the sheath which was guided by fluoroscope. The 5F catheter was then withdrawn. A 7F catheter was inserted through the sheath and advanced to tricuspid valve. Holding the catheter in position, a cutting wire was inserted into the 7F catheter advanced to tricuspid leaflets. Chordae tendineae were engaged by the cutting wire by rotating the catheter towards the ventricular wall and simultaneously advancing the cutting wire in the open position. The cutting wire was withdrawn slightly to check for engagement which can be determined by simple tactile sensitivity. If chordae isolation was confirmed, then the cutting wire was withdrawn further to disrupt the chordae. This approach allows for only 1 chordae disruption at a time so it was repeated multiple times until the desired level of peripheral venous reflux was obtained as determined by duplex and pressure measurements, i.e., until the pressure gradient was between right ventricle and atrium was <2 mmHg. The cutting wire was then withdrawn. A contrast agent (Omnipaque, GE Healthcare, Waukesha, Wis.) was injected from the 7F catheter and fluoroscopy was performed to verify that reflux was occurring from RV and RA. The 7F catheter and sheath were removed and skin puncture closed by pressuring the incision for 10-20 minutes. Animals were allowed to fully recover under the appropriate post-operative pain management.

One of the objectives in this protocol was to study the flow and pressure of the lymph in thoracic duct. Therefore, it is a critical parameter to monitor the flow in walking animals. This was achieved by placing a flow-probe around the TD. Specifically, thoracotomy was operated. A 15 cm incision was operated between 6^{th} and 7^{th} ribs. The ribs were separated to 5 to 8 cm with thoracic retractor. The lung was gently moved by malleable retractor. The thoracic duct was identified and dissected free over approximately 1 cm. Transonic flow probe was placed to thoracic duct and connected to transonic meter. The flow probe was sutured with adjacent tissue to avoid movement as soon as the correct reading was displayed on the flowmeter. The thoracotomy was closed by returning ribs to original positions (removing the retractor), suturing muscle layers (0-0 Gut chrome), pulmonary inflation, suturing subcutaneous connective tissue (0-0 Gut chrome), and subcutaneous suturing (3-0 Proline). The incision and probe cable were protected in a pocket on swine jacket. Animals were allowed to fully recover under the appropriate post-operative pain management.

### C. Hemodynamic and Anatomy

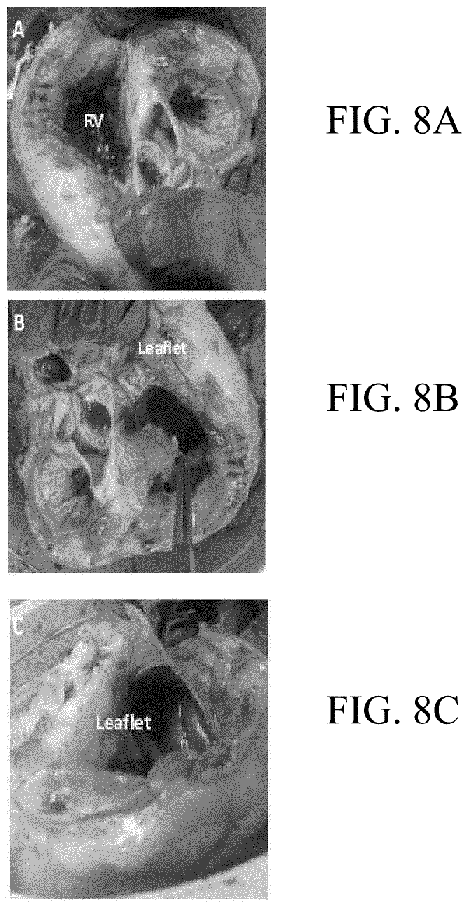

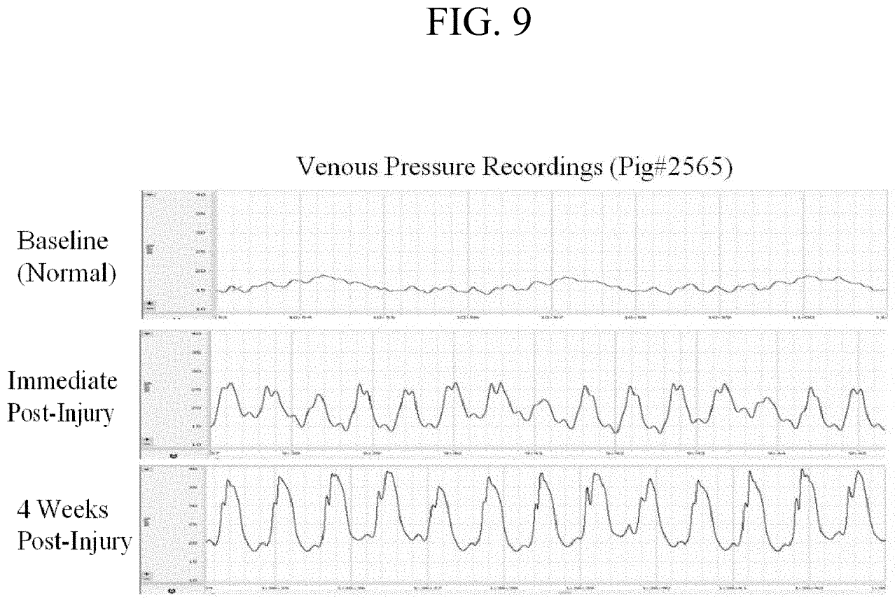

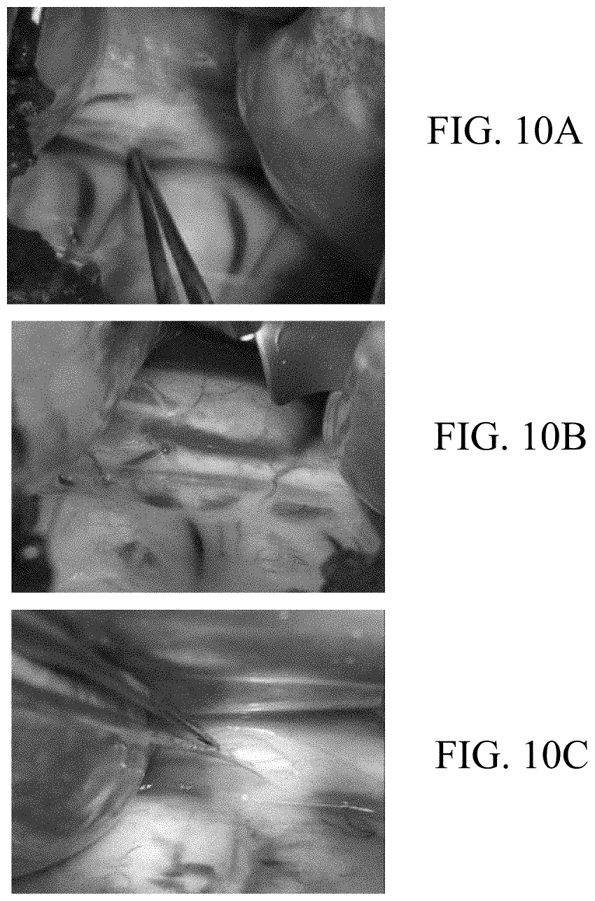

The TCR animal model was successful. Various parameters support the goals of TCR model. The Chordae tendineaes were cut and the leaflets lost the function to maintain one-direction flow (FIG. 8). Right ventricle significantly remodeled (Table 7). Jugular venous pressures were arterialized (FIG. 9). In overview examination, TD was dilated in post tricuspid injury and edema was formed around the aortic-TD sheath (FIG. 10).

The Tricuspid Injury Model is pictured in FIG. 8. The image 8A is the right ventricle (RV). The image 8B shows the tricuspid leaflet at septum where several chordaes were cut and several chordaes still survived. The imaged 8C shows the tricuspid leaflet at anterior, where all chordaes were cut. The leaflet could reverse completely.

TABLE 7
Right Ventricular Hypertrophy

| | | Postop | Heart | LV | RV | | |
|---|---|---|---|---|---|---|---|
| Pig # | PIG ID | (weeks) | (gram) | (gram) | (gram) | RV/LV | ΔIVP (mmHg) |
| 1 | 2324 | 4 | 291 | 142 | 87 | 0.61 | 3 |
| 2 | 2565 | 4 | 283 | 157 | 75 | 0.48 | 4 |
| 1 | 2564 | 4 | 341 | 185 | 101 | 0.55 | 5 |
| 4 | 2498 | 6 | 400 | 195 | 116 | 0.59 | 1 |
| 5 | 2496 | 6 | 398 | 197 | 115 | 0.58 | 3 |
| 6 | 2693 | 6 | 356 | 171 | 109 | 0.64 | 3.9 |
| 7 | 3661 | 12 | 320 | 170 | 90 | 0.53 | 3.8 |
| 8 | 3793 | 12 | 356 | 190 | 108 | 0.57 | 3 |
| 9 | 3823 | 12 | 367 | 179 | 115 | 0.64 | 4 |
| 10 | 5018 | 12 | 433 | 208 | 140 | 0.67 | 5.8 |
| 11 | 5022 | Flow probe failed | — | — | — | — | — |
| 12 | 5684 | 12 | 311 | 153 | 96 | 0.63 | 4 |
| 13 | 5025 | 4 | 251 | 131 | 68 | 0.52 | 5 |
| 14 | 6026 | 4 | 231 | 115 | 67 | 0.58 | 5 |
| 15 | 6078 | 4 | 255 | 135 | 68 | 0.50 | 7 |
| 16 | 6323 | 4 | 301 | 161 | 91 | 0.57 | 5 |
| 17 | 6330 | 4 | 287 | 153 | 77 | 0.50 | 11 |
| 18 | 6331 | 4 | 298 | 153 | 71 | 0.45 | 4 |

| | | | | | | |
|---|---|---|---|---|---|---|
| Mean | 7 | 322 | 165 | 94 | 0.57 | 4.6 |
| SD | 4 | 57 | 25 | 21 | 0.05 | 2.1 |

TABLE 8
Pressure Distribution in the TD (mmHg)

| | | | | | | | | [symbol] |
|---|---|---|---|---|---|---|---|---|
| Pig ID | Postop (wks) | Vein Outlet | ID-Vein function | Aortic Arch | Heart Apex | 10 cm Lower | CC | Pressure |
| 2324 | 4 | 8 | 8 | 11 | 15 | 21 | 22 | 14 |
| 2565 | 4 | 9 | 11 | 13 | 16 | 19 | 22 | 13 |
| 6026 | 4 | 9 | 10 | 13 | 16 | 19 | 19 | 9 |
| 6026 | 4 | 9 | 6 | 8 | 10 | 11 | 12 | 3 |
| 6078 | 4 | 9 | 16 | 16 | 19 | 20 | 20 | 11 |
| 6323 | 4 | 10 | 13 | 11 | 13 | 14 | 16 | 6 |
| 6330 | 4 | 14 | 10 | 12 | 14 | 16 | 16 | 2 |
| 6331 | 4 | 18 | 10 | 12 | 15 | 17 | 15 | [symbol] 3 |
| Mean | | 10.8 | 10.5 | 12 | 14.8 | 17.1 | 17.8 | 6.9 |
| SD | | 3.5 | 3.0 | 2.3 | 2.4 | 3.8 | 3.6 | 5.9 |
| 2408 | 6 | 10 | 10 | 11 | 11 | 14 | 14 | 4 |
| 2496 | 6 | 11 | 5 | 7 | 8 | 8 | 8 | [symbol] 3 |
| 2693 | 6 | 7 | 7 | 12 | 14 | 14 | 15 | 8 |
| Mean | | 9.3 | 7.3 | 10.0 | 11.0 | 12.0 | 12.3 | 3.0 |
| SD | | 2.1 | 2.5 | 2.6 | 3.0 | 3.5 | 3.8 | 5.6 |
| 3661 | 12 | 13 | 15 | 17 | 20 | 22 | 23 | 10 |
| 3793 | 12 | 12 | 14 | 16 | 19 | 22 | 22 | 19 |
| 5018 | 12 | 3 | 4 | 4 | 6 | 10 | 10 | 6 |
| 5684 | 12 | [symbol] | [symbol] | 12 | 14 | 12 | 11 | 5 |

| | | | | | | | |
|---|---|---|---|---|---|---|---|
| Mean | 8.3 | 9.8 | 12.3 | 14.5 | 16.5 | 16.5 | 7.8 |
| SD | 4.9 | 5.6 | 5.9 | 6.9 | 6.4 | 6.9 | 2.6 |

[symbol]

indicates data missing or illegible when filed

FIG. 10 shows the significant dilation of the thoracic duct observed in an animal after 12 weeks post tricuspid injury. In the image 10A, the lymphatic thoracic duct shows less distension in a normal pig. Image 10B shows the lymphatic thoracic duct dilated at 12 wks post tricuspid injury and clearly visible. Image 10C shows edema formed around the aortic-TD sheath.

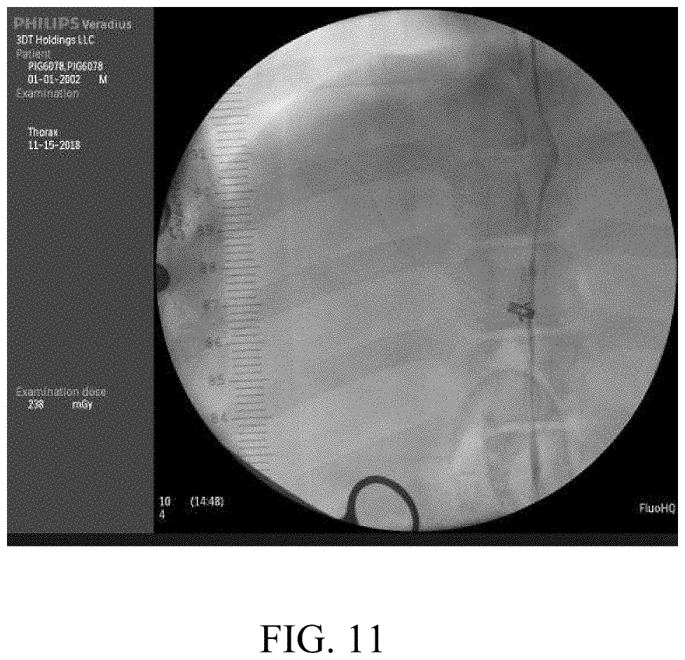

Transonic flow probes were implanted in TDs of three pigs to track chronical flow variations (FIG. 11). TD flow increased almost immediately after tricuspid injury and remained elevated until terminal study (Table 9).

TABLE 9
Chronic Flow (ml/min)

| | | | | | | | Postop 4 | |
|---|---|---|---|---|---|---|---|---|
| | | After | | | Postop 2 | Postop 3 | weeks | |
| Pig ID | Baseline | injury | Postop 1 day | Postop 1 week | weeks | weeks | (terminal) | |
| 3823 | 0.17 | 0.41 | 3.6 | 2.7 | 3.3 | — | — | |
| 5018 | 0.6 | — | 0.3 | 2.7 | 7.2 | — | — | |
| 5022 | 0.15 | — | — | — | — | — | — | |
| 5843 | 0.12 | 1.9 | 2.1 | 3.9 | — | — | — | |
| 6025 | — | — | — | — | — | — | 4.9 | |
| 6026 | — | — | — | — | — | — | 3.3 | |
| 6078 | — | — | — | — | — | — | 3.7 | |
| 6323 | 0.1 | 0.6 | 5.6 | 12.5 | 12.3 | 13.2 | 5.1 | Chronic probe |
| | | | | | | | 6.6 | Acute probe |
| 5330 | 0.1 | 3.5 | 4.7 | 53 | 3.4 | 11.5 | 3.25 | Cronic probe |
| | | | | | | | 3.1 | Acute probe |
| 6331 | 0 | 7.1 | 15.7 | 13.8 | 9 | 7.3 | 4.5 | Chronic probe |
| | | | | | | | 5.8 | Acute probe |

### D. Histology

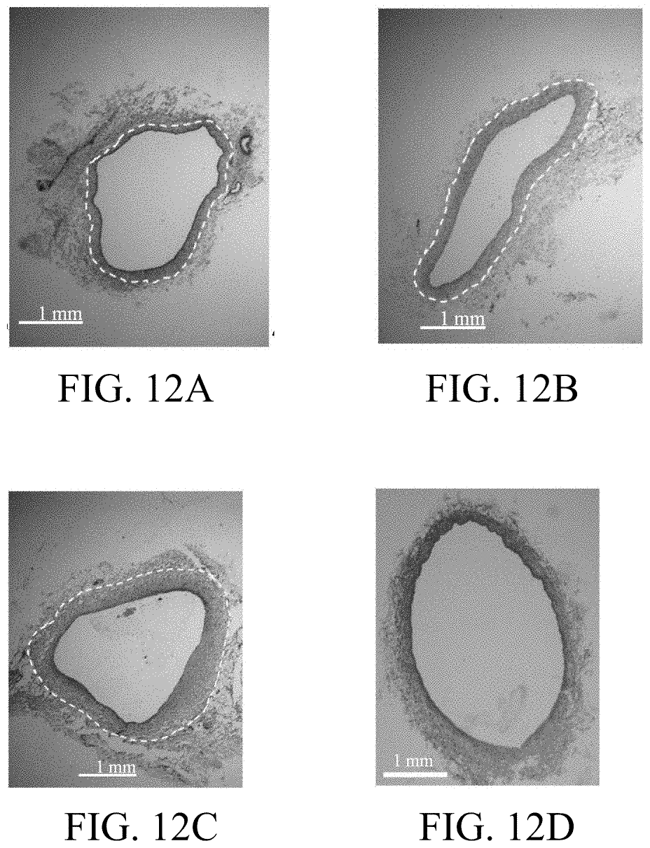

TD was thickened during the remodeling in post tricuspid injury. FIG. 12 shows a thoracic duct enlargement and thickening during the remodeling after tricuspid injury. Image 12A is the control. Image 12B is 4 weeks post injury. Image 12C is 6 weeks post injury. Image 12D is 12 weeks post injury.

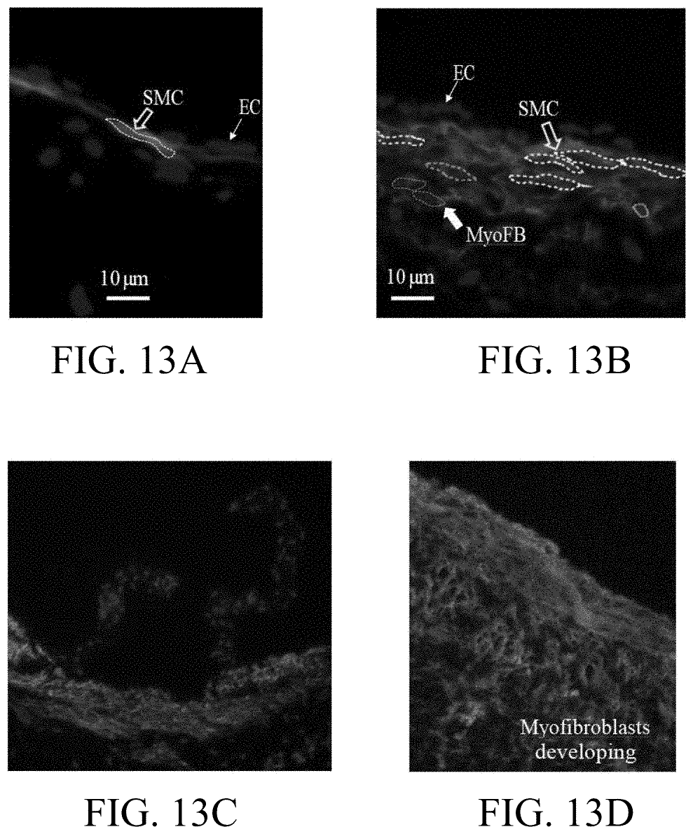

Immunofluorescence microscopy is used to study the structure of TD wall. Anti-alpha-actin (red) visualized smooth muscle cells (SMC) and myofibroblasts (MyoFB) in the TD (FIGS. 13 A&B). We captured TD valve in the segment from tricuspid injury for 6 weeks (FIG. 13 C). Although they were activated, MyoFB did not invade into leaflet of lymphatic valve yet (FIG. 13 C). The smooth muscle layers increased from 1 layer to 3-5 layers post-injury (FIG. 13 and Table 11).

FIG. 13 Antibody of anti-alpha-actin (red) bind to a-actin of smooth muscle cells (SMC). DAPI (Blue) bind to nuclei. Image 13A is the control. One layer SMC is dominant in TD at dorsal endothelial cells (EC). Image 13B shows 6 weeks post injury. White dashed lines indicate SMC nucleus. The image is viewed under an objective 60× lens. MyoFibroblasts were short and wide relatively (blue dash lines). Image 13C shows 6 weeks post injury. The image captured lymphatic valves and viewed under a objective 20× lens. Image 13D shows 12 weeks post injury viewed under an objective 60× lens.

TABLE 10
Histological Evaluation

| Thoracic Duct at | | Media | Wall |
|---|---|---|---|
| Postoperative | Layer | Thickness | Thickness |
| weeks | (account) | (mm) | (mm) |
| Control (n = 7) | 1 to 2 | 7 ± 3 | 106 ± 32 |
| 4 weeks (n = 6) | 2 to 4 + MFB | 19 ± 13 | 439 ± 172 |
| 6 weeks (n = 3) | 3 to 4 + MFB | 22 ± 11 | 462 ± 163 |
| 12 weeks (n = 5) | 3 to 7 + MFB | 24 ± 13 | 537 ± 189 |

### E. Mechanical Testing

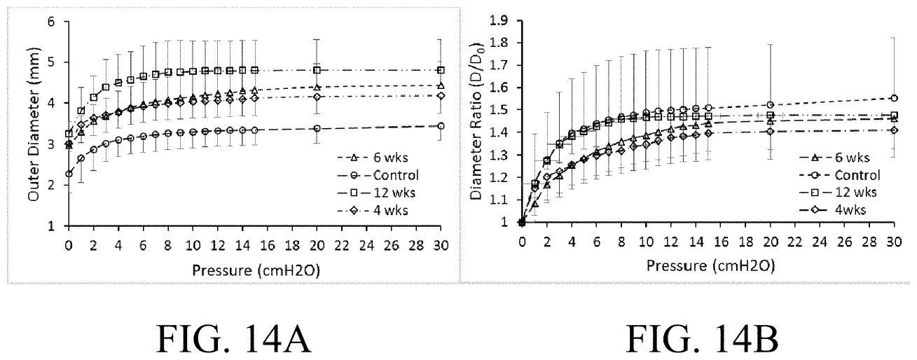

The diameter vs. pressure relationship is represented in FIG. 14A. The diameter ratio vs. pressure is represented in FIG. 14B. As shown in FIG. 14A, the remodeling resulting in the increase in diameter in 4, 6, and 12 weeks post tricuspid injury.

### F. Discussion

Tricuspid regurgitation elevates jugular vein pressure and then the pressure of venous system. It results in an immediate increase in the TD lymph flow. TD lymph flow increased approximate 10 times and reached plateau in 1-3 days and remained for up to 12 weeks. The TD pressure at lymphovenous junction was elevated to ˜11 mmHg at post-op 4 weeks, which is larger than that in normal pigs. The pressure gradient from lymphovenous junction to CC was ˜7 mmHg at post-op 4 weeks, which is slightly smaller than that in normal pigs. The hemodynamic data largely varied due to animal number was too few (only three) to indicate a trend. The TD pressure at lymphovenous junction was pulled back to the level of normal pigs (˜8 mmHg) at post-op 12 weeks. The pressure gradient from lymphovenous junction to CC was slightly smaller at post-op 12 weeks than that in normal pigs. It seems that the TD pressure at lymphovenous junction and CC elevated to adapt the increase in pressure at venous side. However, the pressure gradient did not change very much. In vitro diameter-pressure relationship shows that TD diameter enlarged in post-injury. TD remodeling is observed, e.g., medial smooth muscle layers increased from 1-2 of healthy to 4-5 of post tricuspid injury. We also observed ascites was built up within aortic-TD sheath, though there was not significant ascites accumulation in abdomen. It is well known that CHF developed in human is long periods (years). The period for post-injury might be insufficient in this study to accumulate abdominal ascites.

Section III: Connection to Treatment

The combination of diuretics and vasodilators or angiotensin converting enzyme inhibitors and, in some cases, cardiac inotropic agents are highly effective in achieving the management of congestion and providing significant symptomatic improvement in patients with congestion secondary to CHF. However, renal dysfunction and diuretic resistance often occur in the most severe cases of CHF, which limits the available therapeutic resources to decrease congestion. For patients who do not respond well to or cannot tolerate the above regimen, frequent therapeutic paracentesis (a needle is carefully placed into the abdominal area, under sterile conditions) can be performed to remove large amounts of fluid (up to 4 to 5 liters each time). However, paracentesis may result in complications. Therefore, novel therapeutic decongestive strategies are needed for patients with CHF. In this project, we attempted to establish proof-of-concept for two approaches relying on the Venturi effect. They are designated as balloon concept and fistula concept. An index of the animals and completed experimental protocols is listed in Table 11.

TABLE 11
Phase 3 Animal Study Index

| Number | Animal ID | Date | Study Type | Comments |
|---|---|---|---|---|
| 1 | 6144 | 10/25/2018 | Balloon | Test various balloons. |
| 2 | 6440 | 11/27/2018 | Balloon | Test various balloons. Regional |
| | | | | pressure drop in jugular vein. |
| 3 | 6441 | 11/29/2018 | Balloon | Balloon test finished. TD flow increase. |
| 4 | 6378 | 12/12/2018 | Balloon | Balloon test finished. TD flow increase. |
| 5 | 6551 | 12/21/2018 | Balloon | Animal died during balloon insertion. |
| 6 | 6624 | 01/04/2019 | Balloon | Balloon test finished. TD flow increase. |
| 7 | 6755 | 01/17/2019 | Balloon | 2 weeks tricuspid injury for examination |
| | | | | of balloon efficacy. |
| 8 | 6554 | 01/08/2019 | Fistula | Pig died after fistula for a while. |
| 9 | 6639 | 01/10/2019 | Fistula | Pig died after fistula for a while. |
| 10 | 6871 | 01/25/2019 | Fistula | Pig died after carotid to jugular fistula. |
| 11 | 7042 | 03/07/2019 | Fistula | Pig was alive until experiment ended. |
| | | | | TD flow increased. |
| 12 | 7125 | 03/08/2019 | Fistula | Pig was alive until experiment ended. |
| | | | | TD flow increased. |
| 13 | 7164 | 03/11/2019 | Fistula | No change in TD flow. Lymphovenous |
| | | | | junction was significantly narrow. |

A. Balloon Concept

### (i) Overview

The concerted effort of respiration, lymphatic smooth muscle cells, and lymphatic valves ensure one-way lymph transport to the veins. The failure or compromise of any of these elements or increased production of lymphatic fluid beyond capacity may result in congestion of the lymphatic system that may lead to edema or ascites. We propose a novel mechanical intervention to increase lymph drainage from TD to jugular or subclavian vein. In the proposed novel intervention, a specifically designed balloon catheter would be placed near lymphovenous junction that performs cycles of slow inflation and rapid deflation. The transient pressure in the vein at lymphovenous junction would be lowered by the rapid deflation of the balloon, which may drain extra lymph from TD. The pressure in the vein at lymphovenous junction, however, would be unchanged during the slow inflation of the balloon.

To establish proof of concept, we tested the approach in healthy animals to learn proper procedures then moved to diseased animal with TCR. Due to the stiff learning curve, only one animal (ID 6755) with TCR could be considered in the study. This pig was subjected to TCR for 2 weeks and treated with balloon deflation/inflation in jugular vein at lymphovenous junction.

### (ii) Animal Studies

The pig was anesthetized and lie on the side on the table. Thoracotomy was performed between 6^{th} and 7^{th} to place a flow probe on TD for monitoring TD flow. Abdominectomy was performed to cannulate CC. Contrast was injected from the cannulation for tracing TD and lymphovenous junction. Fluoroscopy was used to access the lymphovenous junction. Volcano wire was used to verify the venous diameter. For the healthy animals, a saline bag (reservoir) was connected to the CC to simulate excess lymph that would be present in congestion (as per Dr. Itknin's suggestion to rule out “Waterfall effect”). To achieve that, inguinal incision was performed to cannulate a saline bag at distal CC. The bag was moved up-and-down the bag to adjust the TD flow such that it is not changed in comparison with the value before the bag connection. The balloon was percutaneously placed into jugular vein. Through trial, we found that CODA 32-LP balloon was most successful to increase TD flow. The balloon was positioned at the junction with the aid of fluoroscope. The balloon was inflated to maximal diameter (˜11 mm) by 2 ml saline injection. The diameter of jugular vein at lymphovenous junction is between 10 to 12 mm determined by both fluoroscopy and IVUS. Therefore, the inflated balloon nearly occupied the lumen of the jugular vein. There was approximate 2 ml volume change after deflation. The inflation/deflation cycles of the balloon were performed for a duration from 30 to 60 minutes. Lymph flow in TD was monitored during the inflation/deflation cycles.

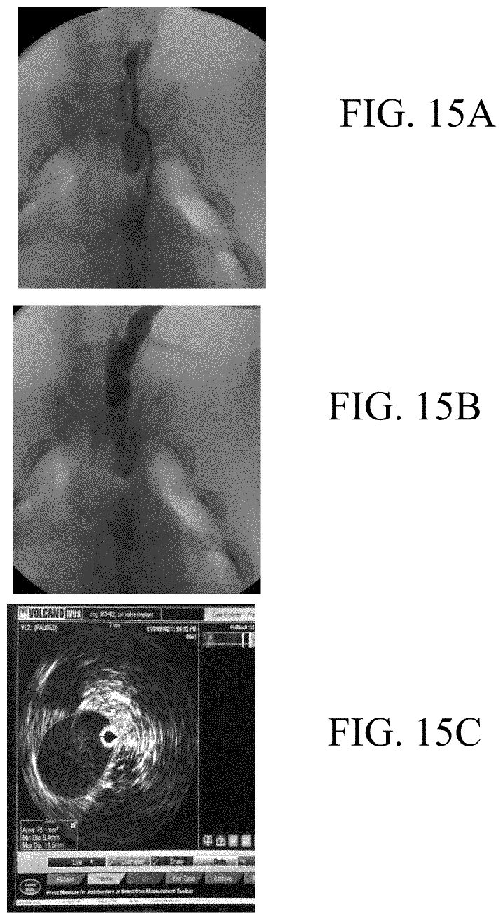

FIGS. 15A-C show images taken during the above localization of the lymphovenous junction. FIGS. 15A and 15B are the tracing of the TD and the jugular vein, respectively, by fluoroscopy, and FIG. 15C shows and IVUS image of the localization procedure.

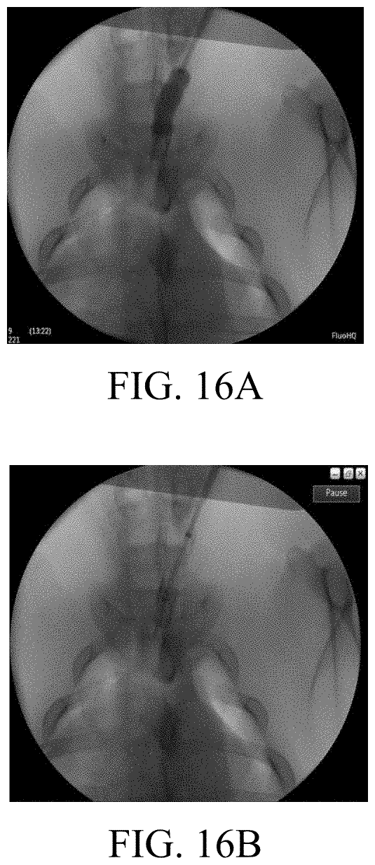

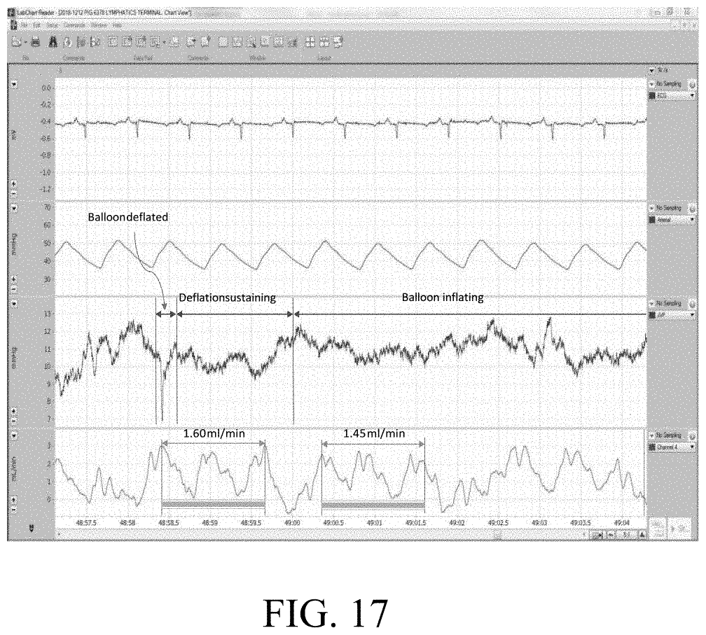

Lymphovenous junctions were generally localized mostly at proximal jugular vein in pigs (FIG. 15). We randomly selected inflation/deflation cycles in the balloon operations to verify whether deflation resulted in TD flow elevation. When balloon was rapidly deflated, a significant pressure decrease in jugular vein was observed (FIG. 17). The decrease in pressure caused TD increase in the following period (FIG. 17). The tracing curves of jugular venous pressure and TD flow shows that the operations of balloon deflation resulted in regional pressure drop and enhanced TD flow (FIG. 16 B, FIG. 17). The operation of balloon inflation did not affect both regional pressure and TD flow, since the inflation was performed slowly (FIG. 16 A, FIG. 17).

Table 12 summarizes TD flows during operations. Of note in Table 12, the delta ratio is the TD flow during deflation minus TD flow at baseline and then divided by TD flow at baseline. If multiplied by 100, it represents the percentage by which TD flow has increased during deflation compared to baseline

TABLE 12
Summary of TD flows for the balloon studies

| | | TD Flow | | |
|---|---|---|---|---|
| | | (m/min) | | |
| Pig ID | Baseline | Inflation | Deflation | ΔRatio |
| 6441 | 0.77 | 0.78 | 1.0 | 0.30 |
| | 1.05 | 1.1 | 1.48 | 0.41 |
| 6378 | 1.44 | 1.45 | 1.6 | 0.11 |
| 6624 | 0.33 | 0.33 | 0.41 | 0.24 |
| | 0.36 | 0.32 | 0.42 | 0.17 |
| Mean | 1 | 0.80 | 0.98 | 0,25 |
| SD | 0.47 | 0.49 | 0.56 | 0.24 |
| 6755 | 3.27 | 3.35 | 3.51 | 0.07 |
| (2 weeks TCR) | 3.06 | 3.28 | 3.52 | 0.15 |
| | 2.4 | 2.62 | 3.28 | 0.37 |
| | 2.5 | 2.87 | 3.27 | 0.31 |
| Mean | 3 | 3.03 | 3.40 | 0.22 |
| SD | 0.42 | 0.35 | 0.14 | 0.14 |

### (iii) Discussion

The animal studies show, in both healthy animals with reservoir and the animal with TCR (2 weeks), that balloon inflation/deflation can increase TD flow, which indicates that balloon concept may relieve the symptoms of CHF by acceleration of draining TD lymph into venous system. However, the balloon needs to be optimized to be suitable for the intervention and to fit the complex anatomy of jugular-subclavian veins to avoid any injury to the veins during balloon inflation/deflation and maximize the efficacy of the intervention. A device needs to be designed for driving the balloon catheter to induce a transient negative pressure at lymphovenous junction. The duration of deflation must be sharply short to transiently lower the pressure. The balloon must be slowly inflated for minimum pressure disturbance in the adjacent space.

B. Fistula Concept

### (i) Overview

The goal of the fistula concept is to lower regional pressure around lymphovenous junction by utilizing the hemodynamic characteristics of artery and vein. When the high-velocity blood in artery is introduced into low velocity of blood in vein (arterial-venous fistula), the jet flow of artery within vein may lower the pressure near the jet. Obviously, pressure near the jet is related to the jet velocity, the angle of arterial to venous flow direction, the distance between the jet and lymphvenous junction, etc. The venous compliance and flow would also affect the pressure near the jet. Our aim is to prove that a fistula near lymphovenous junction can acutely increase lymph flow in TD in animal study.

### (ii) Animal Studies

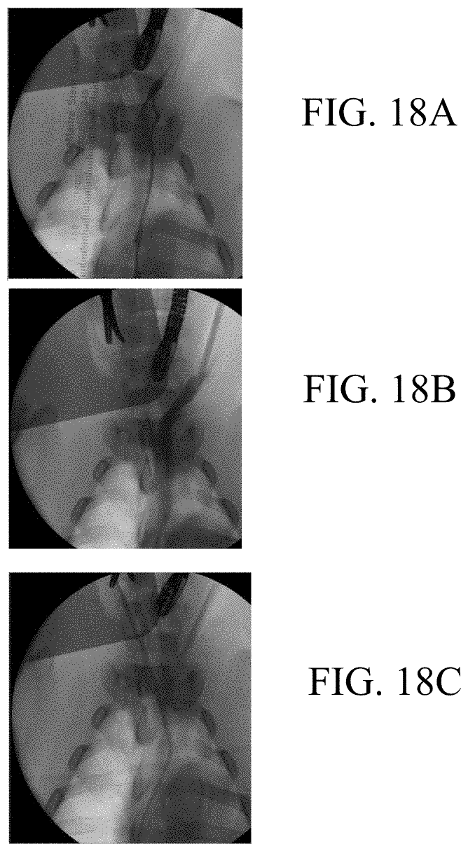

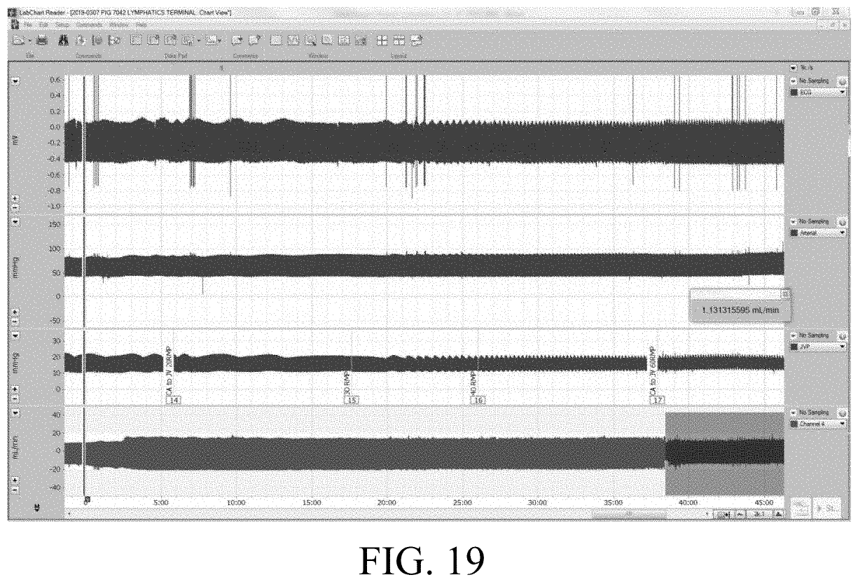

The pig was anesthetized and lie on the side on the table. Thoracotomy was performed between 6^{th} and 7^{th} to place a flow probe on TD for monitoring TD flow. Abdominectomy was performed to cannulate CC. Contrast was injected from the cannulation for tracing TD, and lymphovenous junction. Fluoroscopy was used to find the junction, as in FIG. 18A. FIG. 18B shows the tracing of the jugular vein. A reservoir was included similarly to the balloon studies. Carotid artery and jugular veins were cannulated. An external loop (we call it A-V fistula) connected carotid artery to jugular vein for draining carotid blood into jugular vein at lymphovenous junction as shown in FIG. 18C. A runner pump was lined in the loop to regulate flow rate. A catheter was placed in pulmonary artery/right ventricle to monitor pressure. An esophageal ECHO probe was placed to monitor contraction of right ventricle. Lymph flow in TD was monitored before and after the anastomosis.

The catheter for blood infusion through fistula was placed near to lymphovenous junction (FIG. 18). The increase in TD flow is infusion rate dependent. Table 13 lists the TD flow change at different rates. The delta ratio is the TD flow with fistula minus baseline and then divided by baseline (multiplied by 100 to be expressed in percentage). It represents the percentage by which the TD flow has increased (or decreased if negative percentage) with the fistula compared to baseline (no fistula).

TABLE 13
Summary of TD flows for the fistula studies

| | TD Flow (ml/min) | | | |
|---|---|---|---|---|
| Pig | Base- | Fistula flow | Fistula flow | Fistula flow |
| ID | line | ~30 ml/min/D % | ~60 ml/min/D % | >75 ml/min/D % |
| 6554 | 0.28 | — | 0.48/71% | 0.56/111% |
| 6639 | 0.24 | — | 0.42/75% | 0.65/171% |
| 6871 | 1.48 | — | 1.47/-1% | 1.47/-1% |
| 7042 | 0.45 | 0.81/80% | 0.92/104% | 1.13/148% |
| 7125 | 0.028 | 0.015/-46% | 0.014/-50% | 0.032/14% |
| 7164 | −0.07 | −0.004/-43% | 0.06/-14% | 0.15/114% |
| Mean | 0.401 | 0.27/-3% | 0.56/31% | 0.67/93% |
| SD | 0.56 | 0.466/72% | 0.55/61% | 0.56/71% |

### (iii) Discussion

The acute study shows that an A-V fistula (introduced here as an external loop) can increase TD flow by alteration of hemodynamic environment, which indicates that an A-V fistula concept may relieve the symptoms of CHF by acceleration of draining TD lymph into venous system. We proved that higher perfusion flow rate in an A-V fistula might result in larger lymph flow in the TD. In order to regulate perfusion rate, a runner pump was connected in A-V fistula loop. Three animals died because dysrhythmia was not immediately managed. As dysrhythmia was managed, two pigs were alive when the perfusion flow rates through the A-V fistula were >50 ml/min. When an external pacemaker was applied, a pig did not have dysrhythmia even through the perfusion flow rates through the A-V fistula were >50 ml/min. When the perfusion flow rates through the A-V fistula were <50 ml/min, there was not any complications. However, the perfusion flow rate for actual arterial-venous fistula cannot exceed 50 ml/min in cervical region. Therefore, there is no safety issue for the fistula concept.

Thus the experimental data supports that the Bemoulli principle applies in the veno-lymph junction wherein the acceleration of local blood flow through the veno-lymph junction will have a resulting lowering effect on the local pressure applied to the lymphatic duct. The increase of local blood flow velocity will create a Venturi effect, lowering the pressure applied to the lymphatic duct and allowing the relief of lymphatic congestion caused by disease such as congestive heat failure. This blood flow acceleration can be achieved through methods and devices as described herein.

### Balloon Embodiment

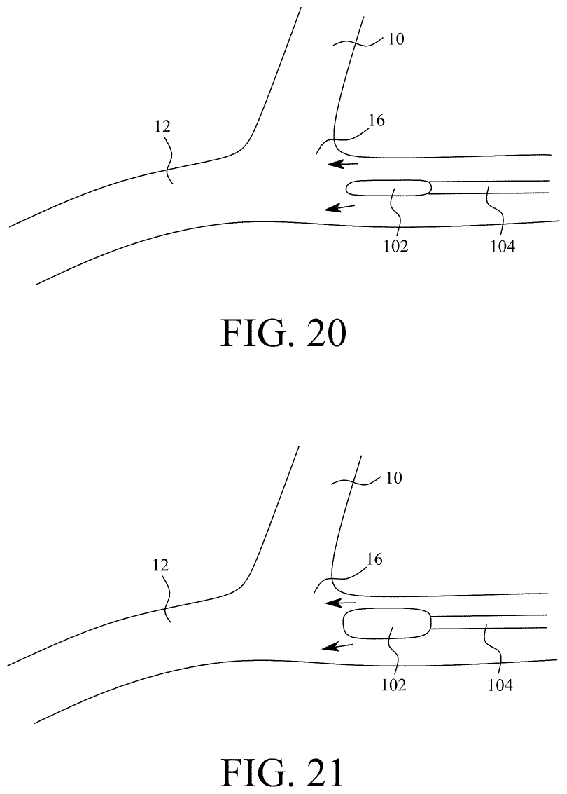

In one embodiment for treating an acute condition of lymphatic congestion, such as in FIGS. 20-21, a catheter 104 having a balloon 102 thereon may be inserted into the subclavian vein 12 near or at the lymphovenous junction 16 to perform cycles of slow inflation and rapid deflation. Arrows indicate the direction of blood flow. This slow inflation of the balloon 102 near the lymphovenous junction 16 locally accelerates the blood flow velocity (i.e., decreases pressure according to Venturi effect) towards the heart during balloon inflation. Preferably inflation is performed at a rate that causes minimal or no pressure disturbance of the adjacent area. That is, the balloon 102 should be inflated sufficiently slowly such that pressure does not rise in the vein. During the balloon deflation process, a suction effect is created since the balloon will deflate faster than the venous wall can respond, lowering the local pressure near the venous-lymph junction thus drawing lymph out of the TD. In this manner, the lymph flow will be towards the heart because this is the path of lower resistance, and because there is a valve at the lymphovenous junction that prevents blood from entering the TD.

The acceleration or increase in velocity of blood flow (accomplished by slowly inflating and rapidly deflating the balloon and the Venturi effect) causes a local decrease in pressure along with the suction effect (which helps draw the lymph from the TD) to relieve the lymph congestion. In other words, this creates an action similar to that of a heart-like device in the vicinity of the lymphovenous junction to increase lymph flow/drainage into the subclavian vein (and then towards the heart) to alleviate lymphatic congestion. The transient pressure in the subclavian vein at the lymphovenous junction may be lowered by the rapid deflation of the balloon, which may drain extra lymph from the TD. In some embodiments, the duration of the balloon deflation may be sharply short sufficient to transiently lower the pressure. However, in an embodiment, the pressure in the vein at the lymphovenous junction would be unchanged during the slow inflation of the balloon, to avoid injury to the veins and for minimum pressure disturbance in the adjacent space.

FIG. 20 shows an embodiment of the present invention. Balloon 102 is disposed on catheter 104 and deployed in the subclavian vein 12. In this embodiment, the balloon 102 is positioned immediately upstream of the veno-lymph junction 16. However, different embodiments may position the balloon elsewhere along the vein, such as further upstream, within the junction, or even downstream from the veno-lymph junction as desired. In FIG. 21, the balloon is partially inflated. As the balloon 102 has been slowly inflated the venous walls have not been affected. However, the velocity of the blood flow downstream from the balloon and moving past the TD 10 is increased due to the narrowed passageway. Thus local pressure at the area of increased acceleration is lowered and lymph is encouraged to drain into the veno-lymph junction 16 and vein 12. Rapid deflation of the balloon 102 would return the balloon to a state as in FIG. 20.

In one embodiment, an off-the-shelf balloon may be used with manual inflation and deflation cycles. The balloon herein may be optimized to fit the complex anatomy of jugular-subclavian veins to avoid any injury to the veins during balloon inflation and deflation and to maximize the efficacy of the intervention/procedure. With the right balloon shape, it is possible to increase TD flow through sustained balloon inflation and deflation. In some embodiments, the balloon may be either compliant or semi-compliant sized to not exert excess pressure on the vein. To prevent excess pressure on the luminal walls of the vein, the balloon may be sized to be no more than 1 mm above the expected, or measured, or estimated, diameter of the vein or vessel. In some alternative embodiments, the balloon may be sized to be no greater than 0.5-1.5 mm larger than the expected diameter of the vein. Additionally, the balloon may be an approximately constant diameter and the inflation lumen of the lumen may be large (likely necessitating a larger catheter) to allow for rapid inflation and deflation of the balloon. The balloon may further have a single chamber and be approximately 2-3 cm long. It should be understood that the sizes herein are exemplary only for purposes of illustration herein and other sizes are also contemplated as being within this scope and/or to amplify particular results and/or for non-standard patient conditions, etc.

In some embodiments, the balloon may be positioned at or near the distal end of the catheter, while the inflation and deflation may be manually controlled via a large inflation lumen at or near the proximal end of the catheter device (such as external to a patient). A device, such as a pump, (not shown) may be positioned near the proximal end of the catheter device for driving the balloon inflation and deflation to induce the transient pressure. In other embodiments, a pump may be programmed to automatically perform a pre-determined cycle of balloon inflation and deflation times. In yet additional embodiments, the pump may be positioned within, or partially within, the catheter device and/or balloon near the distal end of the catheter device. The pump may also be synced to the heart.

As shown by animal studies (see Experimental Results section below), the balloon inflation and deflation cycling can increase or improve TD flow, which indicates that the balloon catheter device may relieve the symptoms of CHF by accelerating drainage of the TD lymph into the venous system.

### Fistula Embodiment

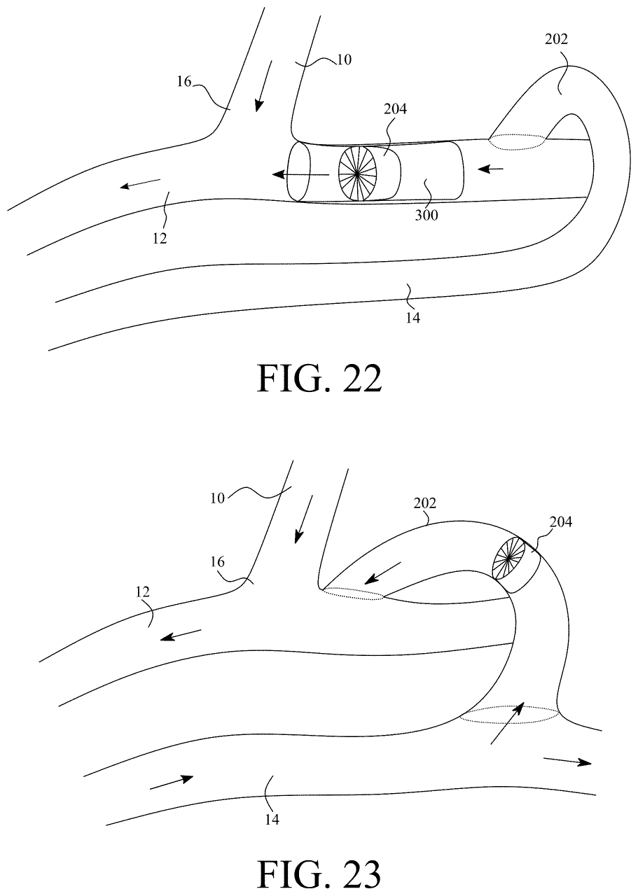

In another embodiment, as pictured in FIG. 22, instead of a balloon, a pump 204, such as an axial flow pump 204 may be inserted into the subclavian vein 12 (via a stent or covered stent 300) and/or an AV fistula 202 (or external loop) may also be created upstream of the veno-lymph junction 16 to locally accelerate venous flow through an arterial jet towards the heart to induce a local pressure drop and facilitate lymph flow out of the TD 10. An axial flow or runner pump 204 may be operably coupled to the subclavian vein similarly to the balloon embodiment described herein above. In this embodiment, the axial flow pump or runner pump may be deployed into a stent, covered stent 300, or similar conduit to hold it securely in place within the subclavian vein. Alternatively, the pump may also be placed within the fistula or elsewhere within the blood flow such that the blood flow velocity near the target lymph duct is increased, such as in FIG. 23. Additionally, the stent containing the pump may remain within the patient for an extended period of time, which may be advantageous for treatment of more chronic cases of lymphatic congestion.

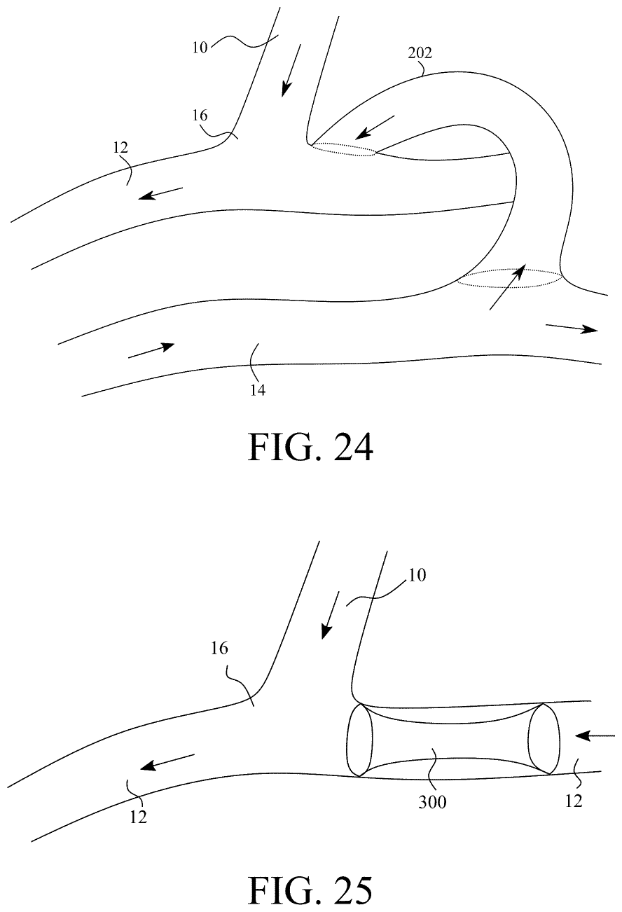

In another embodiment, pictured in FIG. 24, for treating a chronic condition of lymphatic congestion, an AV fistula 202 (i.e., an abnormal connection between an artery and a vein, routing blood flow directly from an artery 14 to a vein 12, bypassing some capillaries) may also be created upstream of the veno-lymph junction 16 to locally accelerate venous flow through an arterial jet towards the heart (for the same reasons as above). In this embodiment, a pump is not used. By the Bernoulli principle accomplishing the Venturi effect, the accelerated flow induces a local pressure drop, which facilitates lymph flow out of the TD.

In yet another embodiment as shown in FIG. 25, a covered stent 300 with a short, narrow, center section (i.e., a “dogbone” shape or configuration) may be placed in the subclavian vein, upstream of the veno-lymph anastomosis (or junction) to create a region of lowered venous pressure at the TD to facilitate lymph drainage therefrom. In this embodiment, the veno-lymph anastomosis can be identified using lymphangiography and then the venous stent may be placed accordingly.

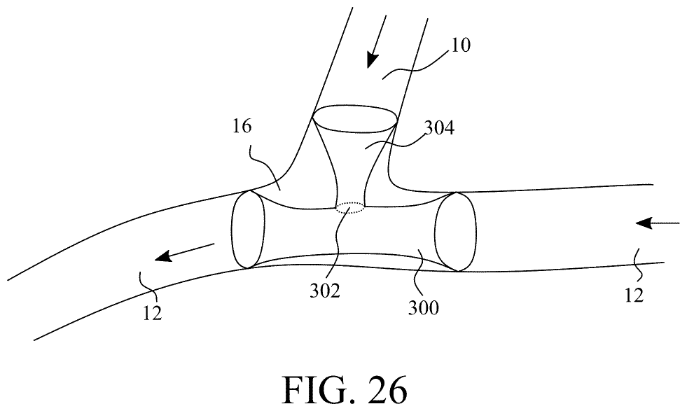

In a related embodiment as shown in FIG. 26, a covered stent may also be deployed directly over the veno-lymph anastomosis (rather than upstream of the anastomosis). In this embodiment, the covered stent may have a side branch 304 which cannulates the veno-lymph anastomosis, and an outlet 302 within the reduced diameter portion of the covered stent, to allow lymph drainage/flow therethrough. In other embodiments, the stent will not have a side branch, but only an outlet aligned with the lymphatic duct.

In the embodiments described, the axial flow pump 204 may be pulsated or cycled in a matter so as to create blood flow acceleration toward the heart, such as similarly to the balloon inflation and deflation described herein above. The axial flow pump (such as a propeller or runner pump) may operate to direct fluid blood flow axially, to provide a higher flow rate, and could even be operated in sync with the right heart. In some embodiments the axial flow pump may be utilized alone, or in combination with the creation of the AV fistula 202.

In another embodiment, an external flow loop may connect the carotid artery to the jugular vein (instead of the subclavian vein) to mimic an AV fistula immediately proximal to the lymphovenous junction to increase TD flow by alteration of the hemodynamic environment. The connection can be made with using off-the-shelf sheaths, catheters, and tubing. Sheaths, pumps and balloons can be also applied as described above, substituting the jugular vein for the subclavian vein.

The AV fistula 202 may be created either surgically or percutaneously using standard methods. The diameter of an exemplary AV fistula may be about 4-6 mm, i.e., not too small to form a thrombus or close off, and not so large as to put an undue load on the heart. There are many examples of AV fistulas in the art, but none used for the lymphatic decongestion purposes disclosed herein. Further, the AV fistula may be positioned or formed such that the area of highest velocity within the fluid is placed to flow directly across the veno-lymph anastomosis.

Some exemplary stents, or covered stent, may consist of a frame, that may be covered with a coating, such as polyethylene terephthalate (PET or PETE) (such as DACRON®, for example), polytetrafluoroethylene (PTFE) (such as TEFLON®, for example), expanded polytetrafluoroethylene (ePTFE) (such as GORE-TEX®, for example), a biological material, etc., and form a “dogbone” configuration once deployed to maintain apposition. In one exemplary embodiment, the stent may comprises a central portion, a first flared portion, and a second flared portion. The configurations (such as diameter and length) of the stent can be predetermined using standard SPY or CT imaging or intraoperatively with vascular imaging (fluoroscopy, intravascular ultrasound (IVUS), brightness (B) mode ultrasound, etc). An exemplary device, stent, or covered stent, and at least one additional item, such as a catheter, a balloon, and/or an axial flow pump, may collectively be referred to herein as a system, or at least a portion of a system. Delivery into a patient may be made percutaneously, as generally referenced above, or laparoscopically, as may be desired for a given procedure.

While various embodiments of devices, methods, and systems for relieving lymphatic congestion have been described in considerable detail herein, the embodiments are merely offered as non-limiting examples of the disclosure described herein. It will therefore be understood that various changes and modifications may be made, and equivalents may be substituted for elements thereof, without departing from the scope of the present disclosure. The present disclosure is not intended to be exhaustive or limiting with respect to the content thereof.

Further, in describing representative embodiments, the present disclosure may have presented a method and/or a process as a particular sequence of steps. However, to the extent that the method or process does not rely on the particular order of steps set forth therein, the method or process should not be limited to the particular sequence of steps described, as other sequences of steps may be possible. Therefore, the particular order of the steps disclosed herein should not be construed as limitations of the present disclosure. In addition, disclosure directed to a method and/or process should not be limited to the performance of their steps in the order written. Such sequences may be varied and still remain within the scope of the present disclosure.

### REFERENCES

1. Itkin, M. Invited Commentary: Lymphatic (or “Forgotten”) Circulation Has to Be Rediscovered. 2016, 2212-2213.
2. Cope, C. Percutaneous thoracic duct cannulation: feasibility study in swine. J. Vasc. Interv. Radiol. 1995, 6, 559-564.
3. Cope, C. Diagnosis and treatment of postoperative chyle leakage via percutaneous transabdominal catheterization of the cisterna chyli: a preliminary study. J. Vasc. Interv. Radiol. 1998, 9, 727-734.
4. Dumont, A. E.; CLAUSS, R. H.; REED, G. E.; TICE, D. A. Lymph Drainage in Patients with Congestive Heart Failure—Comparison with Findings in Hepatic Cirrhosis. N. Engl. J. Med. 1963, 269, 949-952.
5. Witte, M. H.; Dumont, A. E.; Clauss, R. H.; Rader, B.; Levine, N.; Breed, E. S. Lymph circulation in congestive heart failure: effect of external thoracic duct drainage. Circulation 1%9, 39, 723-733.
6. Kinnaert, P. Pressure measurements in the cervical portion of the thoracic duct in man. BJS 1973, 60, 558-561.
7. Shizgal, H. M.; Gutelius, J. R. The role of the subclavian-vein thoracic duct junction in experimental ascites. Ann. Surg. 1%9, 169, 519.
8. Dumont, A. E.; Mulholland, J. H. Flow rate and composition of thoracic-duct lymph in patients with cirrhosis. N. Engl. J. Med. 1960, 263, 471-474.
9. Phang, K. L.; Bowman, M.; Phillips, A.; Windsor, J. Review of thoracic duct anatomical variations and clinical implications. Clin. Anat. 2014, 27, 637-644.
10. Seeger, M.; Bewig, B.; Gunther, R.; Schafmayer, C.; Vollnberg, B.; Rubin, D.; Hoell, C.; Schreiber, S.; Fölsch, U. R.; Hampe, J. Terminal part of thoracic duct: high-resolution US imaging. Radiology 2009, 252, 897-904.
11. Onizuka, M.; FlatebØ, T.; Nicolaysen, G. Lymph flow pattern in the intact thoracic duct in sheep. J. Physiol. 1997, 503, 223-234.
12. Inagaki, M.; Onizuka, M.; Ishikawa, S.; Yamamoto, T.; Mitsui, T. Thoracic duct lymph flow and its driving pressure in anesthetized sheep. Lymphology 2000, 33, 4-11.
13. Tomoyasu, M.; Onizuka, M.; Inagaki, M.; Sato, Y.; Yamamoto, T.; Ishikawa, S.; Mitsui, T. EFFECT OF PHOSPHODIESTERASE ill INHIBITOR (OLPRINONE) ON THORACIC DUCT LYMPH FLOW IN ANESTHETIZED SHEEP WITH EXPERIMENTALLY INDUCED HEART FAILURE BY ENDOTHELIN-1. Lymphology 2002, 35, 144-152.
14. Knott, E. M.; Tune, J. D.; Stoll, S. T.; Downey, H. F. Increased lymphatic flow in the thoracic duct during manipulative intervention. J. Am. Osteopath. Assoc. 2005, 105, 447-456.
15. Lattuada, M.; Hedenstierna, G. Abdominal lymph flow in an endotoxin sepsis model: influence of spontaneous breathing and mechanical ventilation. Crit. Care Med. 2006, 34, 2792-2798.
16. Downey, H. F.; Durgam, P.; Williams Jr, A. G.; Rajmane, A.; King, H. H.; Stoll, S. T. Lymph flow in the thoracic duct of conscious dogs during lymphatic pump treatment, exercise, and expansion of extracellular fluid volume. Lymphat. Res. Biol. 2008, 6, 3-13.
17. Jamalian, S.; Davis, M. J.; Zawieja, D. C.; Moore, J. E. Network Scale Modeling of Lymph Transport and Its Effective Pumping Parameters. PLoS One 2016, 11, e0148384.
18. Cole W R, Witte M H, Kash S L, Rodger M, Bleisch W R, Muelheims G H. Thoracic duct-to-pulmonary vein shunt in the treatment of experimental right heart failure. Circulation. 36(4): 539-43, 1967.

1. A method for reducing pressure at a veno-lymph junction to increase lymph flow from a lymphatic duct and alleviate lymphatic congestion, comprising:
inserting a balloon catheter into a vein of a patient and positioning the balloon near the patient's veno-lymph junction;
slowly inflating the balloon via an inflation lumen coupled to an inflation and deflation source, wherein slow inflation locally accelerates blood flow velocity toward a patient's heart;
rapidly deflating the balloon after the balloon is inflated to create a suction effect to draw lymph out of a lymphatic duct; and
repeating a cycle of slowly inflating the balloon and rapidly deflating the balloon until lymph flow from the lymphatic duct has been increased to alleviate the lymphatic congestion at the veno-lymph junction.

2. The method of claim 1, wherein the method further comprises sizing the vein or veno-lymph junction to size the balloon catheter to be no more than 1 mm greater than an interior diameter of the vein near the veno-lymph junction to avoid exerting excess pressure on the vein.

3. The method of claim 1 wherein the cycle of slowly inflating the balloon and rapidly deflating the balloon is performed automatically by a programmed pump.

4. The method of claim 1, wherein the vein is the subclavian vein and the lymphatic duct is the thoracic duct.

5. The method of claim 1, wherein the step of slowly inflating the balloon is performed at a rate such that the local pressure does not rise.

6. The method of step 3, wherein the balloon is inflated and deflated in sync with the right heart.

7. A method for reducing pressure at a veno-lymph junction to relieve lymphatic congestion, comprising:
forming an arterial-venous (AV) fistula upstream of the veno-lymph junction to locally accelerate blood flow through an arterial jet towards the heart; and
wherein the formation of the AV fistula locally accelerates blood flow at the veno-lymph junction and induces a local pressure drop at the thoracic duct to facilitate lymph flow therefrom to relieve lymphatic congestion.

8. The method of claim 7, further comprising the steps of:
inserting a stent into the subclavian vein near the veno-lymph junction.

9. The method of claim 8, wherein the stent has an axial flow pump disposed therein; and further comprising the step of:
operating the axial flow pump to create blood flow acceleration toward the heart to decrease pressure and increase lymph flow from the thoracic duct and relieve lymphatic congestion.

10. The method of claim 8, wherein inserting the stent comprises inserting a covered stent having a short, narrow, center section and wherein the covered stent is configured for placement within a patient's subclavian vein, upstream of the veno-lymph junction, to create a region of lowered venous pressure at the thoracic duct.

11. The method of claim 8, wherein the inserting the covered stent further comprises a stent having a side branch configured to cannulate the veno-lymph junction and an outlet positioned with the short, narrow, center section, wherein the covered stent is deployed over the veno-lymph junction itself.

12. The method of claim 7, wherein diameter of the AV fistula is 4 mm to 6 mm in diameter.

13. The method of claim 10, wherein the stent is positioned such that a narrowed portion of the stent spans the veno-lymph junction.

14. The method of claim 7 wherein the AV fistula connects the carotid artery and one of either the jugular vein or the subclavian vein.

15. The method of claim 7 wherein the AV fistula is connected immediately proximal veno-lymph junction.

16. A system for reducing pressure at a veno-lymph junction to increase lymph flow from a lymphatic duct and alleviate lymphatic congestion, comprising:
a catheter having a balloon thereon sized for insertion near a patient's veno-lymph junction and configured for inflation via a user at a proximal end thereof;
wherein the balloon is sized to be no more than 1 mm greater than an internal diameter a patient's subclavian vein near the veno-lymph junction to avoid exerting excess pressure on the subclavian vein; and
wherein cycles of slow inflation and rapid deflation of the balloon locally accelerate blood flow velocity towards the heart during the inflation, and creates a suction effect during the deflation, to draw lymph out of the thoracic duct and alleviate lymphatic congestion.

17. The system of claim 16, wherein the balloon further comprises a compliant or semi-compliant balloon having a single chamber and a consistent size.

18. The system of claim 16, wherein the system comprises a manually activated inflation and deflation source.

19. The system of claim 16, wherein the system comprises a programmable inflation and deflation source.

20. The system of claim 16, wherein the programmable inflation and deflation source is programmed for a slow inflation and a rapid deflation.
